(19) United States
(12) Patent Application Publication
(10) Pub. No.: US 2004/0214323 A1
(43) Pub. Date: Oct. 28, 2004

(54) Long lived keratinocytes

(76) Inventors: Marcia Simon, Stony Brook, NY (US); Maja Matic, Bayside, NY (US)

Correspondence Address: FISH & RICHARDSON PC, 225 FRANKLIN ST, BOSTON, MA, 02110, US

(21) Appl. No.: 10/855,309

(22) Filed: May 27, 2004

Related U.S. Application Data
(63) Continuation of application No. 10/154,567, filed on May 24, 2002.

(51) Int. Cl. C12N 5/08; C12N 15/85

(52) U.S. Cl. 435/366; 435/455

(57) ABSTRACT

The invention features methods of producing a preparation of very long lived epithelial cells, e.g., keratinocytes. The method includes providing a human epithelial tissue, e.g., epidermis; isolating at least one keratinocyte clone from the tissue; and determining if the clone is capable of at least 100, 150, 200, 250, 300, 350 or 400 population doublings after isolation from human tissue. Preparations of very long lived keratinocytes and methods of using the keratinocytes are also provided.

### BACKGROUND

All epithelial tissues undergo lifelong cell turnover. Epidermal homeostasis is maintained by stem cells, which by definition have self-renewing capacity that extends to at least the life span of the organism (Lajtha (1979) Differentiation 14: 23-34). Transient amplifying cells, the progeny of stem cells, can divide but appear to have limited proliferative potential, as they are committed to terminal differentiation (Potten (1983) in Stem Cells: Their Identification and Characterization, Churchill Livingston, London, pp. 200-232; Morris et al. (1985) J Invest Dermatol. 84: 277-281; MacKenzie & Bickenbach (1985) Cell Tissue Res 242: 551-556; Potten (1986) Int J Radiat Biol 49: 257-278; Bickenbach (1986) Cell Tissue Kinet 19: 325-333). Their expansion decreases the number of divisions required in the stem cell population and has been proposed as a mechanism to limit accumulation of replicative errors in the stem cell population (Potten & Loeffler (1990) Development 110: 1001-1020; Cairns (1980) Proc R Lond B Biol Sci 208: 121-133).

A major advance in the study of epidermal cells was the development of methods to culture keratinocytes. Using the procedures described by Rheinwald and Green (Rheinwald & Green (1975) Cell 6: 331-343), human keratinocytes can be serially cultivated on a feeder layer of irradiated 3T3 cells. Confluent sheets of keratinocytes, obtained when individual colonies fuse, typically contain 2-4 cell layers with differentiated cells lying above the layer of basal cells. Such cultures have been successfully used for the treatment of extensive full-thickness burn injury and are maintained as permanent wound coverage (Gallico et al. (1984) N Engl J Med 311: 448-451). Cells within the graft maintain the ability to serve as stem cells in vivo and have been shown to express the body-specific markers of the donor (Compton et al. (1998) Differentiation 64: 45-53). Similar results have been obtained in experiments with human xenografts on athymic mice (Kolodka et al. (1998) Proc Natl Acad Sci USA 95: 4356-4361).

### SUMMARY

The invention is based, in part, upon the discovery that -long-lived (LL), moderately long lived (MLL) and very long lived (VLL) human epithelial cells, e.g., keratinocytes, having the potential for long term proliferation can be isolated from human tissue, e.g., skin. The invention features LL, MLL and VLL cells and related methods. Although not wanting to be bound by theory, it is believed that the early cloning of cells from a tissue is important in producing the LL, MLL and VLL cells described herein. As used herein, a “long-lived” or “LL” cell refers to a cell that undergoes 100 or more doublings before entering senescence. A “moderately long-lived” or “MLL” cell refers to a cell that undergoes between 100 and 200 doublings before entering senescence. A “very long lived cell” or “VLL cell” refers to a cell that undergoes at least 200 doublings before entering senescence. In some embodiments, a VLL cell will undergo 250, 300, 350 or even 400 cell doublings, before entering senescence. In some embodiments, an LL, MLL or VLL cell is free of a gross chromosomal abnormality. A preferred LL, MLL or VLL cell is an epithelial cell, e.g., a keratinocyte.

Accordingly, the invention features a method of producing a preparation of epithelial cells, e.g., LL, MLL or VLL cells, e.g., keratinocytes. The method includes providing a source of human epithelial tissue, e.g., epidermis, or mucosal epithelium; isolating at least one clone, e.g., a keratinocyte clone, from the tissue; and determining if the clone is capable of a predetermined number of doublings, e.g., at least 150, 200, 250, 300, 350 or 400 population doublings after isolation from human tissue.

In a preferred embodiment the source is a tissue sample taken from the subject. In other embodiments the source is the subject and the cell is isolated directly from the subject.

In a preferred embodiment, the human tissue is skin, e.g., adult skin.

In a preferred embodiment, the keratinocyte clone is isolated prior to, or prior to a time sufficient for, seven, six, five, four, three or two doublings from the time the sample of human tissue is obtained.

In a preferred embodiment, the keratinocyte clone is isolated, e.g., from a tissue sample or from a subject, before the clone has divided a preselected number of times, e.g., before it has gone through seven, six, five, four, three or two doublings, preferably before two, four or seven doublings. For example, the clone is isolated from a tissue sample before it has divided a preselected number of times, e.g., seven, six, five, four, three or two times, in the period between gathering of the tissue sample and isolation of the clone.

In a preferred embodiment, the keratinocyte clone is isolated directly from the human tissue before the clone has divided once in the period between gathering of the tissue sample and isolation of the clone, e.g., without first passaging the cells.

In a preferred embodiment, determining if the clone is capable of a predetermined number of doublings, e.g., at least 150, 200, 250, 300, 350 or 400 population doublings after isolation from human tissue includes providing a cell from the clone and performing a cell division assay on the provided cell.

In a preferred embodiment, determining if the clone is capable of a predetermined number of doublings, e.g., least 150, 200, 250, 300, 350 or 400 population doublings after isolation from human tissue includes dividing the clone into at least two aliquots and performing serial passaging of the cells of one of the aliquots until the proliferative potential of the cells is exhausted or until the cells undergo a predetermined number of doublings, e.g., 150, 200, 250, 300, 350 or 400 population doublings from the time of isolation from human tissue.

In a preferred embodiment, the cell isolated to form the preparation is free of a gross chromosomal abnormality.

In a preferred embodiment, substantially all of the keratinocytes in the preparation are free of a gross chromosomal abnormality.

In a preferred embodiment, the keratinocyte clone includes an exogenous nucleic acid, e.g., DNA, which causes the production of a protein (e.g., an exogenous regulatory sequence that causes the production of a protein, e.g., an endogenous protein; or an exogenous nucleic acid that encodes a protein). The exogenous nucleic acid can be introduced before or after isolation of the keratinocyte clone. For example, in one preferred embodiment, the exogenous nucleic acid is introduced into a precursor of the keratinocyte clone. In another preferred embodiment, the exogenous nucleic acid is introduced into a cell of the isolated clone. This cell can be recloned to produce an LL, MLL or VLL keratinocyte clone that includes the exogenous nucleic acid. In a preferred embodiment, the exogenous nucleic acid causes the production of a therapeutic protein, e.g., a therapeutic protein described herein.

In some embodiments, the method further includes immortalizing the clone containing the exogenous nucleic acid.

In another aspect, the invention features a method of producing a preparation of keratinocytes which includes providing a human skin sample; isolating at least one keratinocyte clone directly from the skin sample without first passaging the cells; and determining if the clone is capable of a predetermined number of doublings, e.g., at least 100, 150, 200, 250, 300, 350 or 400 population doublings after isolation from human tissue by dividing the clone into at least two aliquots and performing serial passaging of the cells of one of the aliquots until the proliferative potential of the cells is exhausted or until the cells undergo 100, 150, 200, 250, 300, 350 or 400 population doublings from the time of isolation from human tissue.

In a preferred embodiment, the cell isolated to form the preparation is free of a gross chromosomal abnormality.

In a preferred embodiment, substantially all of the keratinocytes in the preparation are free of a gross chromosomal abnormality.

In a preferred embodiment, the keratinocyte clone includes an exogenous nucleic acid, e.g., DNA, which causes the production of a protein (e.g., an exogenous regulatory sequence that causes the production of a protein, e.g., an endogenous protein; or an exogenous nucleic acid that encodes a protein). The exogenous nucleic acid can be introduced before or after isolation of the keratinocyte clone. For example, in one preferred embodiment, the exogenous nucleic acid is introduced into a precursor of the keratinocyte clone. In another preferred embodiment, the exogenous nucleic acid is introduced into a cell of the isolated clone. This cell can be recloned to produce an LL, MLL or VLL keratinocyte clone that includes the exogenous nucleic acid. In a preferred embodiment, the exogenous nucleic acid causes the production of a therapeutic protein, e.g., a therapeutic protein described herein.

In some embodiments, the method further includes immortalizing the clone containing the exogenous nucleic acid.

The invention also includes cell preparations and isolated cells, e.g., LL, MLL or VLL cells or preparations made by a method described herein.

Accordingly, in another aspect, the invention features a preparation of epithelial cells, e.g., keratinocytes, in which substantially all of the colony-forming epithelial cells, e.g., keratinocytes, in the preparation are capable of a predetermined number of doublings, e.g., at least 100, 150, 200, 250, 300, 350 or 400 population doublings after isolation from human tissue.

In a preferred embodiment, the preparation is derived from a clone, e.g., a keratinocyte clone, that is isolated, e.g., from a tissue sample or from a subject, before the clone has divided a preselected number of times, e.g., before it has gone through seven, six, five, four, three or two doublings, preferably before two, four or seven doublings. For example, the clone is isolated from a tissue sample before it has divided a preselected number of times, e.g., seven, six, five, four, three or two times, in the period between gathering of the tissue sample and isolation of the clone.

In a preferred embodiment, the preparation is derived from a clone, e.g., a keratinocyte clone, that is isolated directly from the human tissue, e.g., before the clone has divided once in the period between gathering of the tissue sample and isolation of the clone, e.g., without first passaging the cells.

In a preferred embodiment, the cells are free of a gross chromosomal abnormality.

In a preferred embodiment, cells of the preparation include an exogenous nucleic acid, e.g., DNA, which causes the production of a protein (e.g., an exogenous regulatory sequence that causes the production of a protein, e.g., an endogenous protein; or an exogenous nucleic acid that encodes a protein). The exogenous nucleic acid can be introduced before or after isolation of the parent cell clone, e.g., parent keratinocyte clone, of the preparation. For example, in one preferred embodiment, the exogenous nucleic acid is introduced into a precursor of the parent keratinocyte clone. In another preferred embodiment, the exogenous nucleic acid is introduced into a cell of the preparation. This cell can be recloned to produce an LL, MLL or VLL keratinocyte clone that includes the exogenous nucleic acid. In a preferred embodiment, the exogenous nucleic acid causes the production of a therapeutic protein, e.g., a therapeutic protein described herein.

In some embodiments, the cells or preparation of cells including an exogenous nucleic acid can be immortalized.

In another aspect, the invention features a preparation of LL, MLL or VLL cells, e.g., keratinocytes, obtained from direct cloning of cells taken from a human tissue sample, where the cloning is performed prior to, or prior to a time sufficient for, two cell doublings from the time the human tissue is taken from the human.

In a preferred embodiment the cells are capable of a predetermined number of doublings, e.g., at least 100, 150, 200, 250, 300, 350 or 400 population doublings from the time of isolation from human tissue. The cells are preferably free of a gross chromosomal abnormality.

In a preferred embodiment, the tissue sample is an epithelial tissue sample.

In a preferred embodiment, the cell is a keratinocyte.

In a preferred embodiment, the cloning is performed prior to, or prior to a time sufficient for, one cell doubling from the time the human tissue is taken from the human.

In a preferred embodiment, the cell is capable of at least 100 population doublings from the time of isolation from human tissue.

In a preferred embodiment, the cell is capable of at least 200 population doublings from the time of isolation from human tissue.

In a preferred embodiment, the cell is capable of at least 300 population doublings from the time of isolation from human tissue.

In a preferred embodiment, the cell is capable of at least 400 population doublings from the time of isolation from human tissue.

In a preferred embodiment, cells of the preparation include an exogenous nucleic acid, e.g., DNA, which causes the production of a protein (e.g., an exogenous regulatory sequence that causes the production of a protein, e.g., an endogenous protein; or an exogenous nucleic acid that encodes a protein). The exogenous nucleic acid can be introduced before or after isolation of the cell, e.g., keratinocyte, clone of the preparation. For example, in one preferred embodiment, the exogenous nucleic acid is introduced into a precursor of the parent keratinocyte clone. In another preferred embodiment, the exogenous nucleic acid is introduced into a cell of the preparation. This cell can be recloned to produce an LL, MLL or VLL keratinocyte clone that includes the exogenous nucleic acid. In a preferred embodiment, the exogenous nucleic acid causes the production of a therapeutic protein, e.g., a therapeutic protein described herein. In some embodiments, the cells or preparation of cells including an exogenous nucleic acid can be immortalized.

The invention also includes methods of producing a product, e.g., a therapeutic product, with a preparation or isolated cell described herein, e.g., a preparation or isolated LL, MLL or VLL cell made by a method described herein. LL, ML or VLL cells can be used to provide a therapeutic product to a subject in-vitro, ex vivo, or in vivo.

Accordingly, in another aspect, the invention features a method of producing a product, e.g., a therapeutic polypeptide, protein, RNA, or DNA. The method includes providing a LL, MLL or VLL cell, e.g., keratinocyte, described herein, where the cell includes an exogenous nucleic acid which causes the production of the product; and allowing the cell or a descendant thereof, to produce the product.

In a preferred embodiment, the LL, MLL or VLL cell is isolated directly from a human tissue before the clone has divided once in the period between gathering of the tissue sample and isolation of the clone, e.g.,. without first passaging the cells.

In a preferred embodiment, the product is a therapeutic protein, e.g., Factor VIII, Factor IX, human growth hormone (hGH), erythropoietin (EPO), glucagon -like peptide-1 (GLP-1), α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or biologically active fragment thereof.

In some embodiments, the method further includes immortalizing the cell or preparation of cells including the exogenous nucleic acid.

The invention also features methods of providing a substance with a preparation or isolated cell described herein, e.g., a preparation or isolated cell made by a method described herein. In one embodiment, the invention features a method of producing a product, e.g., a polypeptide, protein, e.g., therapeutic protein, RNA, or DNA. The method includes providing a preparation of LL, MLL or VLL cells, e.g., keratinocytes, e.g., a preparation described herein, wherein substantially all of the colony forming cells of the preparation include an exogenous nucleic acid which causes the production of the product; and allowing the production of the product.

In a preferred embodiment, the invention features methods of providing a substance, e.g., a polypeptide, protein, e.g., therapeutic protein, or RNA, to a subject, e.g., an animal or a human subject. The methods include introducing into the subject a preparation of LL, MLLL or VLL cells or an isolated LL, MLL or VLL cell described herein, e.g., an isolated epithelial cell, e.g., keratinocyte, wherein the epithelial cell is capable of a predetermined number of doublings, e.g., at least 100, 150, 200, 250, 300, 350 or 400 population doublings from the time of isolation from human tissue and, optionally, is free of a gross chromosomal abnormality; and allowing the isolated epithelial cell, e.g., keratinocyte, or a descendent thereof, to produce the substance.

In a preferred embodiment, the cell isolated to form the preparation is free of a gross chromosomal abnormality.

In a preferred embodiment, the substance is a therapeutic protein, e.g., Factor VIII, Factor IX, human growth hormone, erythropoietin (EPO), glucogen-like peptide-1 (GLP-1), or a lysosomal enzyme (e.g., α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase (also known as N-acetylgalactosamine-6-sulfatase), β-galactosidase, N-acetylgalactosamine-4-sulfatase (arylsulfatase B), β-glucuronidase or biologically active fragments thereof).

In another aspect, the invention features a product, e.g., a therapeutic protein, made by the process of: (a) providing an LL, MLL or VLL cell preparation described herein, e.g., an LL, MLL or VLL keratinocyte preparation, where cells of the preparation include an exogenous nucleic acid which causes the production of the product, and where the preparation is capable of a predetermined number of doublings, e.g., at least 100, 150, 200, 250, 300, 350 or 400 population doublings from the time of isolation from human tissue and, optionally, is free of a gross chromosomal abnormality; and (b) allowing the LL, MLL or VLL cell preparation to produce the product.

In a preferred embodiment, the exogenous nucleic acid includes a regulatory sequence that causes the production of a product, e.g., an endogenous protein, e.g., an endogenous therapeutic protein described herein.

In a preferred embodiment, the exogenous nucleic acid encodes a protein, e.g., a therapeutic protein described herein.

In a preferred embodiment, the LL, MLL or VLL cell preparation is allowed to produce the product in vitro. In some embodiments, the LL, MLL or VLL cell or cell preparation is immortalized.

In a preferred embodiment, the LL, MLL or VLL cell preparation is allowed to produce the product in vivo.

In another aspect, the invention features a method of treating a subject, e.g., a human subject, e.g., providing a substance to the subject. The method includes:

(a) identifying a subject in need of a treatment, e.g., in need of a substance;

(b) optionally, providing an interim treatment to the subject, e.g., by administering a needed substance to the subject by a means other than by the administration of a preparation of cells that have been confirmed to be LL, MLL or VLL cells;

(c) providing a preparation of LL, MLL or VLL cells or an isolated LL, MLL or VLL cell, e.g., a preparation or cell described herein, which produces the substance; and (d) introducing the preparation of cells or the isolated cell into the subject, thereby treating the subject. Embodiments of the method allow immediate interim treatment of a subject while the LL, MLL or VLL cells are obtained or confirmed as being LL, MLL or VLL.

In a preferred embodiment, the subject is treated for a deficiency of any of the following substances: Factor VIII, Factor IX, human growth hormone, erythropoietin (EPO), glucogen-like peptide-1 (GLP-1), or a lysosomal enzyme (e.g., α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase or biologically active fragments thereof).

In a preferred embodiment, the interim treatment includes a administering the substance by a means other than gene or cell therapy, e.g., by administering a purified preparation of the substance, e.g., a polypeptide, e.g., purified Factor VIII, Factor IX, human growth hormone, erythropoietin (EPO), glucogen-like peptide-1 (GLP-1), or a lysosomal enzyme (e.g., α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase or biologically active fragments thereof).

In a preferred embodiment, the interim treatment includes administering a cell from a clone which, at the time of administration, has not been confirmed as being LL, MLL or VLL. This embodiment can include testing the clone to determine if it is LL, MLL or VLL, e.g., by performing a cell division assay described herein.

In a preferred embodiment, the interim treatment is other than administration of the substance, e.g., the interim treatment can be surgery, radiotherapy, immunotherapy, or a change in diet or environment.

In a preferred embodiment, the interim treatment is continued for a period of time after the LL, MLL or VLL cell or preparation is administered. For example, if the interim treatment is administration of a purified polypeptide, the purified polypeptide is administered, or maintained at a therapeutic level, until after any of: the LL, MLL or VLL cells are administered, the LL, MLL or VLL cells are confirmed to produce the substance at a therapeutic level, or the LL, MLL or VLL cells are confirmed to be LL, MLL or VLL. In some embodiments, the LL, MLL or VLL cells are confirmed to be LL, MLL or VLL by performing a cell division assay, e.g., on an aliquot of the LL, MLL or VLL cells.

In a preferred embodiment, the method includes introducing into the subject a first epithelial cell, e.g., keratinocyte; allowing the first epithelial cell, e.g., keratinocyte, or a descendent thereof, to produce the substance; further introducing into the patient an LL, MLL or VLL preparation or isolated cell described herein, e.g., an isolated epithelial cell, e.g., keratinocyte, wherein the second epithelial cell is capable of a predetermined number of doublings, e.g., at least 100, 150, 200, 250, 300, 350 or 400 population doublings after isolation from human tissue and, optionally, is free of a gross chromosomal abnormality; and allowing the isolated epithelial cell, e.g., keratinocyte, or a descendent thereof, to produce the substance. This method can be used, e.g., to provide a “bridging” therapy, by providing a subject with a substance (by cell therapy) during the time that it takes for an LL, MLL or VLL cell to be identified for subsequent cell therapy.

In a preferred embodiment, the cell isolated to form the LL, MLL or VLL preparation is free of a gross chromosomal abnormality.

In another aspect, the invention features methods of treating a disorder, e.g., a disorder disclosed herein, in a subject, e.g., an animal or a human subject. The methods include identifying a subject in need of a product, e.g., a protein or RNA; and introducing into the subject a preparation of or an isolated LL, MLL, or VLL cell described herein, e.g., an isolated epithelial cell, e.g., keratinocyte, wherein the epithelial cell includes an exogenous nucleic acid which causes the production of the product in an amount sufficient to ameliorate a symptom of the disorder, and wherein the epithelial cell is capable of a predetermined number of doublings, e.g., at least 100, 150, 200, 250, 300, 350 or 400 population doublings from the time of isolation from human tissue and, optionally, is free of a gross chromosomal abnormality.

In a preferred embodiment, the cell isolated to form the preparation is free of a gross chromosomal abnormality.

In a preferred embodiment, the disorder is hemophilia A, hemophilia B, anemia, diabetes, or a lysosomal storage disease, e.g., Fabry Disease, Gaucher disease, Hurler-Scheie syndrome, Hunter syndrome, Sanfilippo A syndrome, Sanfilippo B syndrome, Sanfilippo C syndrome, Sanfilippo D syndrome, Morquio A syndrome, Morquio B syndrome, Maroteaux-Lamy syndrome, or Sly syndrome.

In another aspect, the invention features methods of treating a disorder, e.g., a disorder disclosed herein, in a subject, e.g., an animal or a human subject. The methods include identifying a subject in need of the product; introducing into the subject a first epithelial cell, e.g., keratinocyte, wherein the first epithelial cell includes an exogenous nucleic acid which causes the production of the product in an amount sufficient to ameliorate a symptom of the disorder; and further introducing into the patient a second isolated epithelial cell, e.g., a preparation or isolated LL, MLL or VLL cell described herein, e.g., a keratinocyte, wherein the second epithelial cell includes an exogenous nucleic acid which causes the production of the product in an amount sufficient to ameliorate a symptom of the disorder, and wherein the second epithelial cell is capable of a predetermined number of doublings, e.g., at least 100, 150, 200, 250, 300, 350 or 400 population doublings after isolation from human tissue and is free of a gross chromosomal abnormality.

In a preferred embodiment, the cell isolated to form the preparation is free of a gross chromosomal abnormality.

In another aspect, the invention features a bank or other plurality of epithelial cell, e.g., keratinocyte, preparations, wherein substantially all of the colony forming epithelial cells in each of the plurality are capable of a predetermined number of doublings, e.g., at least 100, 150, 200, 250, 300, 350 or 400 population doublings after isolation from human tissue and are free of a gross chromosomal abnormality, e.g., a chromosomal deletion, rearrangement, or duplication. In a preferred embodiment, the cell isolated to form the preparation is free of a gross chromosomal abnormality.

In another aspect, the invention features methods of selecting a very long lived epithelial cell, e.g., a keratinocyte, for transplant into a subject, e.g., an animal or human subject. The methods include:

providing information about the subject;

providing information about a preparation of epithelial cells, e.g., keratinocytes, or the individual from which it is derived, from a bank of epithelial cell preparations including a plurality of epithelial cell preparations, wherein substantially all of the colony forming epithelial cells in each of the plurality are capable of a predetermined number of doublings, e.g., at least 100, 150, 200, 250, 300, 350 or 400 population doublings after isolation from human tissue and are free of a gross chromosomal abnormality, e.g., a chromosomal deletion, rearrangement, or duplication, each of the plurality of epithelial cell preparations having a different genotype; and

comparing the information, e.g., genotype, haplotype, or blood type information, about the subject to the information about the preparation of epithelial cells. Preferably, the cell isolated to form the preparation is free of a gross chromosomal abnormality.

In a preferred embodiment the method includes introducing the selected preparation or cell into the subject.

In another aspect, the invention features methods of providing a preparation or isolated cell described herein, e.g., an LL, MLL or VLL keratinocyte preparation, to a subject, which methods include providing a putative LL, MLL or VLL epithelial cell, e.g., keratinocyte, preparation; determining if the putative epithelial cell preparation is LL, MLL or VLL; and administering the LL, MLL or VLL epithelial cell, e.g., keratinocyte, preparation to the subject, e.g., an animal or human subject.

In another aspect, the invention features a method of identifying a marker, e.g., a gene marker or a physical marker, that correlates with the ability of an epithelial cell, e.g., a keratinocyte, to undergo a predetermined number of doublings, e.g., at least 100, 150, 200, 250, 300, 350 or 400 population. The method includes providing a preparation of epithelial cells, e.g., keratinocytes, wherein substantially all of the colony forming epithelial cells in the preparation are capable of a predetermined number of doublings, e.g., at least 100, 150, 200, 250, 300, 350 or 400 population doublings; selecting a putative marker; and determining if the marker correlates with the ability of an epithelial cell, e.g., keratinocyte, to undergo a predetermined number of doublings, e.g., at least 100, 150, 200, 250, 300, 350 or 400 population doublings. In one embodiment, determining if a maker correlates with the ability of an epithelial cell, e.g., keratinocyte, to undergo a predetermined number of doublings, e.g., at least 100, 150, 200, 250, 300, 350 or 400 population doublings, includes comparing any of: a gene expression profile, physical characteristic, or protein activity profile, of an LL, MLL or VLL cell described herein with a reference cell, e.g., a cell known to not be an LL, MLL or VLL cell.

In another aspect, the invention features a method of maintaining a population of colony forming epithelial cells, e.g., keratinocytes, wherein substantially all of the epithelial cells can divide a predetermined number of doublings, e.g., at least 100, 150, 200, 250, 300, 350 or 400 times after isolation from human tissue. The method includes providing a preparation or isolated cell described herein, e.g., an isolated epithelial cell which has the ability to double a predetermined number of times, e.g., at least 100, 150, 200, 250, 300, 350 or 400 times after isolation from human tissue or a preparation of epithelial cells wherein substantially all of the colony-forming epithelial cells in the preparation are capable of a predetermined number of doublings, e.g., at least 100, 150, 200, 250, 300, 350 or 400 population doublings after isolation from human tissue; and culturing the epithelial cell or preparation of epithelial cells under conditions suitable to maintain the ability of the epithelial cells to proliferate.

In another aspect, the invention features a method of maintaining a population of colony forming epithelial cells, e.g., keratinocytes, wherein substantially all of the epithelial cells can divide a predetermined number of doublings, e.g., at least 100, 150, 200, 250, 300, 350 or 400 times after isolation from human tissue. The methods include providing a preparation or isolated cell described herein, e.g., an isolated epithelial cell which has the ability to double a predetermined number of times, e.g., at least 100, 150, 200, 250, 300, 350 or 400 times after isolation from human tissue or a preparation of epithelial cells wherein substantially all of the colony-forming epithelial cells in the preparation are capable of a predetermined number of doublings, e.g., at least 100, 150, 200, 250, 300, 350 or 400 population doublings after isolation from human tissue; and culturing the epithelial cell or preparation of epithelial cells under conditions suitable to maintain at least 5%, 10%, 15% of the epithelial cells in a non-differentiated state.

In another aspect, the invention features a method of providing a keratinocyte system, e.g., an artificial skin system, for evaluating a treatment. The method includes providing a keratinocyte system made by the following method: supplying a preparation or isolated cell described herein, e.g., an isolated keratinocyte which has the ability to double a predetermined number of doublings, e.g., at least 100, 150, 200, 250, 300, 350 or 400 times after isolation from human tissue or a preparation of keratinocytes wherein substantially all of the colony forming keratinocytes in the preparation are capable of a predetermined number of doublings, e.g., at least 100, 150, 200, 250, 300, 350 or 400 population doublings after isolation from human tissue; culturing the isolated keratinocyte or keratinocyte preparation to form a skin substitute; applying the skin substitute to a subject; and exposing the skin substitute to the treatment and evaluating the effect of the treatment.

An “immortalized cell” or “immortalizing a cell” refers to the establishment of a non-senescing cell line from a parent cell. Immortalizing a cell can include altering the parent cell's growth properties. For example, a cell, e.g., an LL, MLL or VLL cell described herein, e.g., an LL, MLL or VLL keratinocyte described herein, can be immortalized by, e.g., infection with a viral oncogene, e.g., HPV-16 or SV-40; transformation with a cellular oncogene; fusion with a growth-deregulated cell, e.g., a cancer cell; activation of telomerase activity; or exposure to a mutagen. Methods for the immortalization of cells and culture of immortalized cells are known in the art (see, e.g., Culture of Immortalized Cells, R. Freshney and M. Freshney (Eds.)), 1996, Jossey-Bass, NY. An immortalized LL, MLL or VLL cell, e.g., an immortalized LL, MLL or VLL cell including an exogenous nucleic acid that causes the production of a therapeutic protein, can be used, e.g., in the in vitro production of a therapeutic protein.

An “exogenous nucleic acid” (e.g., DNA) refers to a nucleic acid introduced into a subject cell or a parent cell of a subject cell. An exogenous nucleic acid can be human or non-human. For example, human DNA can be exogenous to a human cell if it is introduced into the human cell.

A “preparation” of cells (e.g., a preparation of LL, MLL or VLL cells) is a preparation of cells in which substantially all of the colony-forming cells in the preparation exhibit a preselected property. In a preferred embodiment, the property is the ability to divide at least 100, 150, 200, 250, 300, 350 or 400 times. In another preferred embodiment, the property is the absence of a gross chromosomal abnormality. “Substantially all” of the colony-forming cells means at least 60% of the colony-forming cells in a preparation. In some embodiments at least 70, 80, 90% of the colony-forming cells, more preferably at least 95%, 97%, 99% of the colony forming cells or more, up to and including 100% of cells, will have the preselected property.

As used herein, a factor is “exogenous” to a given cell if it is not normally produced by that cell.

As used herein, the term “epithelial cell” means that a cell isolated from epithelial tissue, e.g., from epithelial mucosa or from skin. Epithelial cells of the basal epidermal layer express, e.g., cytokeratins 5 and 14, along with α₆β₄ integrins. Epithelial cells of the suprabasal epidermal layer express, e.g., cytokeratins 1, 2e and 10.

As used herein, the term “single-cell suspension” means a suspension of cells in a liquid wherein substantially all the cells are suspended in the liquid as single cells and are not adherent with another cell. That is, the cells cannot be further dissociated by enzymatic digestion or pipetting. A single cell suspension is most often, but not necessarily, made by enzymatic digestion of a tissue sample or cell culture.

“Substantially all” the cells means at least 60%. In some embodiments substantially all the cells can be at least 70%, 80%, 90% of cells, more preferably at least 95%, 97%, 99% of cells or more, up to and including 100% of cells.

As used herein, a cell “clone” is a group of cells derived from successive divisions of an individual cell.

The term “isolating a clone” refers to the process whereby an individual cell or cell colony, derived from successive division of a single cell, is separated from surrounding cells or colonies.

As used herein, the term “passaging” refers to transferring a cell or cells from a first growth environment to a second growth environment, wherein the cell density of the second is less than that of the first.

As used herein, the term “serially passaging” refers to the process of passaging cells two or more times.

As used herein, an “amount sufficient to ameliorate the symptoms” of a disease or disorder refers an amount of a therapeutic gene product produced by a genetically modified LL, MLL or VLL cell. An amount of a therapeutic gene product sufficient to ameliorate the symptoms of a disease or disorder will vary with the nature of the disease or disorder being treated, but may be determined by monitoring the symptoms being treated. According to the invention, symptoms are ameliorated if the severity of the symptoms is lessened by at least 10%. In some cases, the severity of the symptoms may be lessened by at least 25%, preferably by 50%, 75%, 90% or more, up to and including 100% reduction of symptoms, relative to the severity of symptoms before treatment.

As used herein, a cell that is “free of a gross chromosomal abnormality” is a cell that has a normal karyotype. A normal karyotype means that each of the chromosomes has the standard G-banded pattern on a metaphase chromosome spread. A metaphase chromosome spread is typically visualized and evaluated by Giemsa staining.

The term “treating” or “treatment” as used herein includes preventative (e.g., prophylactic), palliative and curative treatment. Improvement in a disease condition or symptom as a result of the methods of the invention can be evaluated by a number of methods known to practitioners in the art.

The invention provides methods for isolating human LL, MLL, and VLL epithelial cells, e.g., LL, MLL, and VLL keratinocytes. In some embodiments, cells of the invention have the proliferative potential useful to maintain a graft for the lifetime of the recipient. The invention also provides LL, MLL, and VLL cells and methods of using the cells for the provision of therapeutic gene products. The LL, MLL, and VLL cells are also useful for the preparation of auto- and allo-grafts for wound healing. The LL, MLL, and VLL cells described herein are also useful for in vitro assays designed to determine the effects of various compositions or treatments on normal proliferating human skin cells. Such assays allow, for example, for the prediction of harmful effects of various agents on the skin without the need for animal models.

The engraftment of LL, MLL, and VLL human keratinocytes onto wound sites allows long term cell replacement and/or the delivery of therapeutic gene products. Such gene products may be used to treat both genetic deficiencies affecting the skin and systemic genetic deficiencies.

In addition to the requirement for long-term proliferative capacity, and normal karyotype, LL, MLL, and VLL epithelial cells useful for the delivery of therapeutic gene products, wound healing and other therapeutic applications are preferably responsive to normal growth controls and are not immortalized. The cells are preferably not tumorigenic and preferably have normal growth factor requirements.

All scientific literature and patent references referred to herein are incorporated herein in their entirety by reference. The details of one or more embodiments of the invention are set forth in the accompanying drawings and the description below.

Other features, objects, and advantages of the invention will be apparent from the description and drawings, and from the claims.

### DESCRIPTION OF DRAWINGS

FIGS. 1A-B shows data plots of average CFE versus the number of generations before senescence for keratinocyte clones from adult female and male donors.

FIGS. 2A-B, shows the CFE at passage 2 plotted versus the number of generations before senescence for keratinocyte clones from adult female and male donors.

### DETAILED DESCRIPTION

The invention is based in part upon the discovery that non-immortalized, non-tumorigenic human epithelial cells, e.g., keratinocytes, with the potential for long term proliferation (e.g., proliferation for more than 100, 150, 200, 250, 300, 350 or 400 population doublings) can be isolated from human tissue, e.g., skin. Human LL, MLL and VLL epithelial cells disclosed herein have sufficient proliferative potential to maintain a graft for a long period of time, and in some cases, over the lifetime of a graft recipient. LL, MLL, and VLL epithelial cells can be isolated from an individual and used for autologous transplantation into the same individual, thus reducing the potential for immune rejection of grafted material. Such autologous transplantation is useful, for example, for cosmetic or reconstructive procedures, for wound healing, and/or for the delivery of therapeutic gene products.

Methods of Preparing Long Lived Keratinocytes

LL, MLL and VLL epithelial cells can be isolated according to the following protocol.

1. Providing a Human Epithelial Tissue

Tissue can be isolated from essentially any human epithelia, e.g., from adult epidermis, e.g., skin. A preferred location is one having relatively little exposure to the sun, e.g. the inner aspect of the upper arm. Following the cleansing of the donor site (usually with BETADINE™, although other material can be used if it is properly rinsed), the area can be rinsed with sterile saline. A biopsy (e.g., a 4-8 mm punch or equivalent biopsy) is then taken and placed into isotonic buffer (e.g., DMEM) for transport to the laboratory.

The biopsy is washed with an isotonic buffer (for example, phosphate buffered saline, DMEM, or Hanks' buffered saline) to ensure removal of blood products or other contaminants. Any subcutaneous fat can be removed, e.g., surgically. If it is desirable to remove blood products, several more washings can be carried out.

2. Isolating a Keratinocyte Clone from the Human Epithelial Tissue

Early cloning, preferably direct cloning, of the cells from the human tissue can be important for the selection and identification of LL, MLL and VLL cells. This is because the number of cells derived from any single cell of the tissue is a function of both colony forming efficiency (CFE) and population doubling time (PDT). Therefore, cells with the shortest PDT and greatest CFE will overtake the population prior to senescence (see Example 1). However, as the data described herein will show (see Example 2), a short PDT and large CFE can be independent of proliferative potential because there is no significant correlation between proliferative potential and CFE or PDT. Therefore, cloning of LL, MLL and VLL cells from the human tissue is preferably performed prior to a time sufficient for seven, four, or two, population doublings from the time the tissue sample is obtained. More preferably, cloning of cells from the human tissue is performed directly from a single cell suspension of cells of the tissue sample, e.g., as follows.

The washed biopsy can be minced into small, e.g., 1-4 mm², pieces and incubated with enzymes that catalyze the separation of dermis and epidermis. Incubation is usually carried out overnight at 4° C. with DISPASE II™ (2.5 mg/ml, enzyme available from Roche Molecular Biochemicals). If the biopsy is received early in the day the incubation can be carried out at 37° C. for 2-4 hours. Other enzymes, e.g., thermolysin, can be used to separate dermis and epidermis. The amount of incubation time depends upon the time required to easily remove the epidermis from the underlying tissue.

Once the epidermis is removed, it is rinsed several times, and placed in a proteolytic solution, e.g., trypsin/EDTA (0.06%/0.01% in phosphate buffered saline, respectively), and incubated for a time and at a temperature sufficient to allow disaggregation of the epithelium into a single cell suspension, e.g., the tissue can be incubated at 37° C. for about 15-30 minutes. During this time the tubes can be agitated. The trypsin or other proteolytic solution can then be neutralized with 5% fetal bovine serum or with trypsin inhibitor (10× molar excess) and the cells can be harvested by a low speed centrifugation (<800×g).

To isolate individual clones directly from the tissue sample (now suspended cells), the suspended cells, e.g., keratinocytes, should be plated out at a density sufficiently dilute such that distinct and separable colonies can grow from each cell. As the colony forming efficiency (CFE: [#colonies formed which are >2 mm by 14 days]/[#keratinocytes plated]) of the keratinocytes is not known at the time of this initial plating, several dilutions can be made. For example one could make up sets of 5-10 P100 tissue culture dishes, each containing 100, 500, 1000, 2000, and 10000 keratinocytes. This ensures obtaining sufficient numbers of plates containing distinct and separable colonies. For efficient isolation of LL, MLL and VLL epidermal cell clones, at least 10 colonies can be assessed. Preferably, 50-100 colonies are assessed. For plating, cells can be added either to tissue culture plates already seeded with feeder cells, e.g., lethally irradiated (6000 rads of γ irradiation) 3T3 cells or plated onto tissue culture plastic together with feeder cells, e.g., lethally irradiated 3T3 cells. If an irradiator is not available, the 3T3 cells may be treated with approximately 5 μg/ml mitomycin C to prevent further proliferation (Macpherson & Bryden (1971) Exp Cell Res 69: 240-241); this compound must be washed out prior to use of the cells. The 3T3 cells should cover a portion, e.g., approximately ⅓ of the surface of the plate (i.e. 10⁶ cells per P100 plate).

The cultures are incubated to allow separate colonies (clones) from a single cell to grow to sufficient size for passage (e.g., 50-200 cells per colony). For example, the cultures can be incubated at 37° C. in 7.5% CO₂ (5-10% is usually acceptable) in serum containing keratinocyte medium for 5-10 days, changing the medium every 2-3 days. Medium and culture conditions for growth and passage of keratinocytes and 3T3 cells are known in the art and can be carried out, e.g., as described by Randolph and Simon (1993) J Biol Chem 268:9198-9205 (DMEM: Ham's F12 with adenine (1.8×10⁻⁴ M) in a 3:1 v/v ratio, with 1000 units/ml penicillin, 1 mg/ml streptomycin, 0.4 μg/ml hydrocortisone, 5 μg/ml insulin, 10 ng/ml epidermal growth factor, and 1.2×10⁻¹⁰ M cholera toxin).

Individual colonies (clones) can picked and grown up as follows. Medium is removed, the cultures are washed with EDTA (0.01% in PBS) and 3T3s are released after incubation for about 5 min with EDTA. Cloning rings can then be placed around each colony to be harvested. 20-50 μl of trypsin/EDTA are added and the plate is incubated for 15-20 minutes at 37° C. These are conditions usually required to release keratinocytes from the substratum and from each other. The trypsin is neutralized with 5% fetal bovine serum and each cell isolate is transferred into a single well of a 6 well plate (or into an equivalent size tissue culture plate) containing lethally irradiated 3T3 cells (3T3 coverage is equal to ˜⅓ of the surface).

Although the method described here involves removal of 3T3 cells prior to harvesting clones, this is not an absolute requirement.

The cultures are then incubated as above and medium is changed every 3-4 days.

When the clone has grown to a colony size equivalent to 50-250 cells (usually 50-75% confluence reached by about 5-10 days post plating), cells are harvested using trypsin/EDTA and passaged for growth in mass culture and, optionally, for CFE determination. For mass culture using either P100s or T80s, 10⁶ lethally irradiated 3T3 cells can be used together with, e.g., 2×10⁵ keratinocytes. For CFE determinations, 100 keratinocytes can be plated. However, dependent upon the CFE, adjustments must be made in the number of keratinocytes plated in mass culture and for CFE determination. When CFEs are low (1-10%), accurate CFE determination can require the plating of at least 100, preferably 200-1000 keratinocytes. Similarly, when CFE is in the range of 1%-10%, passage for mass culture can require an increase in the number of keratinocytes plated. For example if the CFE is 1% it is preferable to plate at least 10⁵ keratinocytes. If this is not done, subcloning of individual variants may occur. In addition, it may be important to avoid plating very dense cultures of growing keratinocytes in the early passage cultures, since keratinocytes can produce autocrine factors.

The time at which a culture is passaged is not simply dependent upon the percent confluence. It is also dependent upon colony size. For passage, the colony size is preferably about 200 cells or less and the culture is preferably no more than 75% confluent.

3. Identification of LL, MLL and VLL Keratinocyte Clones

Once a clone is provided, e.g., as described above, a determination can be made as to whether the clone is capable of at least 100, 150, 200, 250, 300, 350 or 400 population doublings, and is, therefore, an LL, MLL or VLL keratinocyte clone. The determination can be made, e.g., by performing a cell division assay on the clone. For example, a cell division assay can involve (a) dividing the clone into at least two aliquots, (b) storing a first aliquot, e.g., by freezing it, and (c) and performing serial passaging of the cells of a second aliquot, until a determination of its proliferative potential can be made. The frozen first aliquot then provides an early passage source of the identified LL, MLL or VLL clone. Such an assay can be performed as follows.

Beginning with the first mass culture (i.e., after the first passage of an isolated clone into mass culture), a first aliquot of a size sufficient to ensure the viability of the clone upon thawing can be frozen for each clone. Methods of freezing cells for storage are known in the art. For example, U.S. Pat. No. 4,940,666 teaches a medium preparation specifically useful for frozen storage of viable human keratinocytes. At least about 5×10⁵ cells per aliquot, and preferably about 1×10⁶ cells in a volume of about 1 ml, can be frozen to ensure that there will be viable cells upon thawing for re-culture. The first aliquot of the clone can be frozen or otherwise stored for future use and a second aliquot of the clone can be serially passaged until senescence. A clone that undergoes 100 or more doublings before entering senescence is identified as an LL clone. If the clone undergoes between about 100 and 200 doublings before entering senescence, it is identified as an MLL clone. If the clone undergoes at least 200 doublings before entering senescence, it is identified as a VLL clone. Thus, once a clone is identified as an LL, MLL or VLL keratinocyte, one can go back to an early passage frozen stock of that clone and expand it for therapeutic or other use. In addition to freezing a first aliquot at the time of the first mass culture of the clone, one can optionally freeze an aliquot of the clone at each passage or less frequently, preferably about every 4th passage.

In another instance, a determination can be made that a keratinocyte clone is a LL, MLL or VLL clone by the identification of a marker which is correlated with the ability of a cell to undergo 100, 150, 200, 250, 300, 350 or 400, population doublings. Examples of markers are, but are not limited to, e.g., an mRNA or a protein whose expression is correlated with the ability of a cell to undergo at least 100, 150, 200, 250, 300, 350 or 400, population doublings; or a physical characteristic of a cell or a cell colony, whose presence or absence is correlated with the ability of a cell to undergo at least 100, 150, 200, 250, 300, 350 or 400, population doublings. Determining if a maker correlates with the ability of an epithelial cell, e.g., keratinocyte, to undergo a predetermined number of doublings, e.g., at least 100, 150, 200, 250, 300, 350 or 400 population doublings, can include comparing an LL, MLL or VLL cell with a non-LL cell. For example, the determination can include comparing any of: a gene expression profile, physical characteristic, or protein activity profile, of an LL, MLL or VLL cell described herein with a reference cell, e.g., a cell known to not be an LL, MLL or VLL cell.

4. Characterization of LL, MLL and VLL Cells

In one embodiment, LL, MLL and VLL cells made by the methods disclosed herein, e.g., LL, MLL or VLL keratinocytes, are non-immortalized, and non-transformed. Methods to evaluate immortalization or transformation are known in the art. For example, the cells described herein can be evaluated for one or more of: tumor formation in nude mice; anchorage independent growth; or growth factor requirements, e.g., the requirement for EGF, a minimum serum concentration, and the impact of growth inhibitors such as TGFβ1. Exemplary methods for these analyses are known in the art. For example, in vitro and in vivo models for transformation can be carried out as described in Boukamp et al. (1985) Cancer Res 45:5582-5592. Non-immortalized cells are valuable, e.g., for use in cell therapy in humans. Preferred LL, MLL and VLL cells for use in the cell therapeutic and cell implantation methods described herein are non-immortalized.

In some embodiments, an LL, MLL or VLL keratinocyte is free of a gross chromosomal abnormality often found in immortalized keratinocytes, e.g., trisomy 8 (Baden et al. (1987) J Invest Dermatol 89(6):574-579); duplication of the long arm of chromosome 8 (U.S. Pat. No. 5,989,837); loss of the p arms of chromosome 8 and 10, del(5)(q13), and del(18)(q12) (Hukku and Rhim (1993) Cancer Genet Cytogenet 68:22-31); or i(6p) and i(8q) (Rice et al. (1993) Mol Biol Cell 4:185-194). A determination can be made that an LL, MLL or VLL keratinocyte clone is free of a gross chromosomal abnormality by karyotype analysis. The normal karyotype for human somatic cells is 23 pairs of chromosomes (22 homologous pairs and one pair of sex chromosomes) or 46 total chromosomes. In addition to the appropriate chromosome or chromosome pair number, a normal karyotype means that each of the chromosomes has the standard G-banded pattern on a metaphase chromosome spread. The preparation of metaphase chromosome spreads and subsequent karyotype analysis is known in the art. An example of the steps involved in classical metaphase spread preparation and analysis is presented below. In addition to the classical method, which uses Giemsa staining to visualize the fixed chromosomes (hence, the term “G-banding”), other methods use, for example, fluorescent staining to visualize the chromosome bands.

Classical metaphase chromosome spreads can be prepared according to the method of Seabright (Seabright (1971) Lancet 2:971-972), essentially as follows. Log-phase cultures are treated with 50 ng/ml colcemid to arrest cells in metaphase. The cells are released from the culture plates with trypsin and centrifuged. After removal of the medium and trypsin, the cells are suspended in a hypotonic 75 mM KCl solution for 20 minutes, then fixed with 3:1 methanol/acetic acid with three changes of fixative. Fixed cells are dropped onto glass slides. Slides are allowed to stand for two weeks, then lightly trypsinized and stained with Giemsa stain. Chromosomal identities are determined by photographing the stained chromosome spreads, cutting out the individual chromosome images and aligning the homologous chromosome pairs for band-to-band comparisons. Gross chromosomal alterations or abnormalities (e.g., a chromosome deletion, duplication, trisomy, amplification, aneuploidy, or rearrangement, e.g., translocation, inversion, or insertion, are apparent to one of skill in the art).

One can also examine the nature of the cells by re-culturing an early-passage (e.g., passage number less than or equal to 5) frozen sample of an LL, MLL or VLL cell clone for extended passages. If the re-cultured cell has the same characteristics with regard to long proliferative lifespan (i.e., at least 100, 150, 200, 250, 300, 350 or 400 cell doublings) and normal karyotype, this procedure provides strong evidence that the proliferative phenotype is not the result of accumulated mutations selected for during culture. Testing of this type can provide additional support for the non-immortalized nature of the LL, MLL and VLL cells.

In some embodiments, one can deliberately immortalize a subject LL, MLL or VLL cell or cell preparation. An immortalized LL, MLL or VLL cell or cell preparation is useful, e.g., as a factory for a therapeutic protein. Immortalization can be performed by, e.g., infecting a subject cell with a viral oncogene, e.g., HPV-16 or SV-40; transforming a subject cell with a cellular oncogene; fusing a subject cell with a growth-deregulated cell, e.g., a cancer cell; activating telomerase activity in a subject cell; or by exposing a subject cell to a mutagen. Methods for the immortalization of cells and culture of immortalized cells are known in the art (see, e.g., Culture of Immortalized Cells, R. Freshney and M. Freshney (Eds.)), 1996, Jossey-Bass, NY. An immortalized LL, MLL or VLL cell, e.g., an immortalized LL, MLL or VLL cell including an exogenous nucleic acid that causes the production of a therapeutic protein, can be used, e.g., in the in vitro production of a therapeutic protein.

5. Culture and Maintenance of Human Keratinocytes.

Isolated human keratinocyes can be maintained in culture according to the methods of Randolph and Simon (1993) J Biol Chem 268: 9198-9205, the entire content of which is hereby incorporated by reference. Basically, human epidermal keratinocytes isolated as described in detail herein are grown in disposable plastic tissue cultureware. All cultures have a layer of feeder cells, e.g., proliferatively inactivated 3T3 cells, either pre-seeded on the dish or added at the time of addition of the keratinocytes.

Details of cell passage methods used during the isolation of LL, MLL or VLL epithelial cells are presented herein. For routine passage and expansion of keratinocytes, cells can be passaged at about 70% confluence by treatment with 0.1% trypsin plus 5×10⁻³ M glucose and 5×10⁻⁴ M EDTA. Passaging refers to the process wherein colonies in tissue culture are released from their support, suspended as a single cell suspension, and a portion of the suspension (up to and including all of the cells) is placed in culture in a fresh tissue culture dish. Because most normal cell types cease or slow division or begin differentiation in culture when they are in contact on all sides with other cells (known as contact inhibition), the process of passaging cells serves to maintain cultured cells at a cell density that promotes active cell division. Passaging cells is also useful to expand the number of cells for therapeutic or other purposes. Serial passaging refers to the process of passaging cells repeatedly each time the cells of the previous passage attain a colony size of about 50 to 200 cells per colony, always maintaining a confluence of less than or equal to about 75%. Serial passage involves the adjustment of the number of cells plated at each passage, using the CFE calculated for cells of the previous passage, such that an approximately constant number of colonies per plate is maintained throughout the serial passages.

Standard basal keratinocyte growth medium can be used. For example, a 3:1 (v:v) mixture of Dulbecco's Minimal Essential Medium (DMEM) and Ham's F12 containing adenine (1.8×10⁻⁴M adenine), 1000 units/ml penicillin, 1 mg/ml streptomycin, 0.4 μg/ml hydrocortisone, 5 μg/ml insulin, 10 ng/ml epidermal growth factor, and 1.2×10⁻¹⁰M cholera toxin. For stock culture maintenance, and passaging, cells can also be grown in the basal medium described, supplemented with 5% fetal bovine serum (FBS). Serum lots can tested for colony forming efficiency prior to use in the culture of cells in order to standardize the medium from lot to lot. Serum lots can be tested by comparison of the growth characteristics of keratinocytes in an existing lot with the characteristics of such cells in the new lot. One, two, three, or more, passages can be performed in the process of serum lot testing, with CFE determined at each passage. The new lot of serum can be accepted for use if the CFE at each passage is greater than or equal to that of cells passaged in parallel in the old lot of serum. For these studies, it is recommended that one always use aliquots of a single batch of frozen keratinocytes, in order to minimize batch-to-batch variations in the test cultures.

3T3 cells for use in generating feeder layers can be maintained in serum-containing medium (e.g., DMEM with 10% FBS) according to methods known in the art. Where desired, 3T3 feeder cells can be removed from keratinocyte co-cultures by a 10 minute incubation at 37° C. with phosphate-buffered saline (PBS, 2.7×10⁻³ M KCl, 1.5×10⁻⁷ M KH₂PO₄, 0.14 M NaCl, 8.1×10-3 M Na₂HPO₄, pH 7.4) supplemented with 5×10⁻⁴ M EDTA.

CFE can be determined by plating 100-1000 keratinocytes on P100 tissue culture plates seeded with 1×10⁶ lethally irradiated 3T3 cells. The formula for CFE is as follows: CFE=((# of colonies >2 mm after two weeks)/(# of cells plated))×100%.

Cumulative Cell Output (CCO) for a clonal isolate is a measure of the total number of cells arising from a cell clone before that clone reaches senescence. CCO can be calculated using the cell counts obtained at each passage throughout the replicative lifespan of the cell, adjusted to reflect the number of cells that would arise if every cell of every passage were re-plated. The formula used to determine CCO is as follows: 2^{[1n(cell Output)-1n(cell input)(CFE)]/1n2}.

Tissue Grafts

LL, MLL and VLL cells can be used for tissue grafts, using the methods described herein, either from the individual to receive the graft (autologous graft or autograft) or from another individual of the same species (allo-graft). For most purposes, including wound healing and the delivery of therapeutic gene products (explained herein), it is preferred that the cells are autologous.

LL, MLL and VLL cells can be used to generate artificial skin for transplantation to an individual in need of such cells (e.g., for wound healing) or in need of a gene product made by those cells. The LL, MLL and VLL cells used for tissue grafting can be genetically engineered cells or non-genetically engineered cells. There are a number of known methods of generating skin equivalents including cultured human keratinocytes. Various methods exist by which LL, MLL and VLL epithelial cells can be put into a form that may be administered to a patient and that permits the engraftment of the cells.

For example, U.S. Pat. No. 6,039,760, incorporated herein by reference, teaches a composite including two collagen layers, one of which contains fibroblasts, and an upper keratinocyte layer. The fibroblasts in the method disclosed in the '760 patent may be autologous to the individual. The keratinocytes are taught to be derived from neonatal foreskin, but may be replaced by the very long lived epidermal cells of the present disclosure.

As another example, U.S. Pat. No. 5,693,332, incorporated herein by reference, teaches human keratinocytes supported on a hydrophilic polyurethane membrane.

As another Example, U.S. Pat. No. 5,610,007, incorporated herein by reference, teaches methods of making chimeric sheets of epithelial cells, the sheets including both autologous and either allogeneic or xenogeneic epidermal keratinocytes.

LL, MLL and VLL cells can be used in therapeutic preparations for a wide range of clinical applications, including, for example, coverage of burns, venous leg ulcers, diabetic ulcers, pressure ulcers and dermatological and other surgery wounds, and coverage of wounds at skin graft donor sites.

One can determine the success of cell grafting using LL, MLL and VLL cells by analyzing a biopsy of the graft site after a given amount of time. The histology of the grafted cells and their progeny can be examined in comparison to that of tissues from non-grafted sites. Preferably, LL, MLL and VLL cells will persist at the graft site. A graft may be considered successful if, after 2 weeks, 3 months or longer, preferably a year or more, and more preferably 5 years or more, a decade or more, or even the natural lifespan of the recipient, LL, MLL and VLL cells are still present and continue to proliferate and produce differentiated keratinocytes.

Production of Therapeutic Proteins

The LL, MLL and VLL cells described herein can be used to produce proteins, e.g., therapeutic proteins. In preferred embodiments, the LL, MLL and VLL cells described herein can be genetically modified, e.g., transfected, to include an exogenous nucleic acid which causes the production of a protein, e.g., a therapeutic protein. The exogenous nucleic acid can encode the therapeutic protein, or it can be an exogenous nucleic acid that acts to activate an endogenous coding sequence. Examples of therapeutic proteins than can be produced in the genetically modified cells include, e.g., insulin, low density lipoprotein (LDL) receptor, Factor VIII, Factor IX, human growth hormone (hGH), erythropoietin (EPO), glucagon -like peptide-1 (GLP-1), and lysosomal enzymes (e.g., α-galactosidase, glucocerebrosidase, α-L-Iduronidase, iduronate-2-sulfatase, Heparan-N-sulfatase, α-N-acetylglucosaminidase, acetyl CoA:α-glucosaminide acetyltransferase, N-acetylglucosamine-6-sulfatase, galactose-6-sulfatase, β-galactosidase, N-acetylgalactosamine-4-sulfatase (arylsulfatase B), β-glucuronidase) or biologically active fragments thereof.

In one embodiment, the genetically modified LL, MLL or VLL cells can be used to produce the protein in cell culture (in vitro). The protein can then be isolated from the cells or their culture media and administered to a subject in need of the protein, e.g., a subject who suffers from a deficiency in the protein. In another embodiment, the genetically modified LL, MLL or VLL cells can be implanted into a subject, e.g., in a tissue graft or in a biocompatible matrix, and allowed to produce the protein in-vivo in the subject. Detailed description of these methods is provided below.

Exogenous DNA

Exogenous DNA incorporated into subject cells, e.g., LL, MLL or VLL cells, can be a DNA which encodes a sequence which causes or alters the production of a gene product, or a portion thereof. The product can be useful to treat an existing condition, prevent it from occurring, or delaying its onset. Exogenous DNA refers to DNA introduced into a subject cell or a parent cell of a subject cell. An exogenous DNA can be human or non-human DNA. For example, human DNA can be exogenous to a human cell if it is introduced into the human cell.

In some preferred embodiments, DNA incorporated into subject cells, e.g., LL, MLL or VLL keratinocytes, can be an entire gene; a coding sequence of a gene, encoding an entire desired protein; or a portion thereof which encodes, for example, the active or functional portion(s) of the protein. The protein can be, for example, a hormone, a cytokine, an antigen, an antibody, an enzyme, a clotting factor, a transport protein, a receptor, a regulatory protein, a structural protein, or a protein which does not occur in nature. The DNA may also encode an RNA or an active or functional portion(s) thereof. The DNA can be produced using genetic engineering techniques or synthetic processes. The DNA introduced into the LL, MLL or VLL keratinocytes can encode one or more therapeutic proteins. After introduction into the LL, MLL or VLL keratinocytes, the exogenous DNA can be stably incorporated into the recipient cell's genome (along with the additional sequences present in the DNA construct used), from which it is expressed or otherwise functions. In other cases, the exogenous DNA can exist episomally within the LL, MLL or VLL keratinocytes.

In preferred embodiments, the subject cells, e.g., LL, MLL or VLL keratinocytes, can be genetically engineered to contain an exogenous DNA sequence which includes a regulatory sequence. Examples of such regulatory sequences include one or more of: a promoter, an enhancer, an intron, an untranslated sequence (UAS), a scaffold attachment region or a transcription binding site. The exogenous DNA sequence can be targeted (e.g., by homologous recombination techniques) to result in the targeted insertion of the regulatory sequence of the DNA sequence, placing a targeted endogenous gene under its control (for example, by insertion of either a promoter or an enhancer, or both, upstream of the endogenous gene or regulatory region). Optionally, the targeted insertion of the regulatory sequence can simultaneously result in the deletion of an endogenous regulatory sequence, such as the deletion of a tissue-specific negative regulatory sequence, of a gene. The targeted insertion of the regulatory sequence can replace an existing regulatory sequence; for example, a tissue-specific enhancer can be replaced by an enhancer that has broader or different cell-type specificity than the naturally-occurring elements, or displays a pattern of regulation or induction that is different from the corresponding nontransfected or noninfected cell. In this regard, the naturally occurring sequences are deleted and new sequences are added. In some cases, the endogenous regulatory sequences are not removed or replaced but are disrupted or disabled by the targeted insertion, such as by targeting the exogenous sequences within the endogenous regulatory elements. The targeted insertion of a regulatory sequence by homologous recombination can result in a LL, MLL or VLL cell expressing a therapeutic protein which it does not normally express. In addition, targeted insertion of a regulatory sequence can be used for cells which make or contain the therapeutic protein but in lower quantities than normal (in quantities less than the physiologically normal lower level) or in defective form, and for cells which make the therapeutic protein at physiologically normal levels, but are to be augmented or enhanced in their content or production. Examples of methods of activating an endogenous coding sequence as described are disclosed in U.S. Pat. No. 5,641,670; U.S. Pat. No. 5,733,761; U.S. Pat. No. 5,968,502; U.S. Pat. No. 6,200,778; U.S. Pat. No. 6,214,622; U.S. Pat. No. 6,063,630; U.S. Pat. No. 6,187,305; U.S. Pat. No. 6,270,989; and U.S. Pat. No. 6,242,218, the contents of which are incorporated herein by reference.

Transgenes can be driven by a promoter or promoter/enhancer combination expressed in epithelial cells, e.g., in differentiated keratinocytes. The gene regulatory elements can be cell-type specific if so desired, or they can be expressed in a less restricted manner. For example, expression may be driven by the promoters of keratinocyte-specific genes, including cytokeratin promoters or other promoters involved in keratinization, e.g., acidic (type I) cytokeratin 10 promoter, or a keratin promoter as described in, e.g., Leask et al. (1990) Genes Dev 4:1985-98 and Vassar et al. (1989) Proc Natl Acad Sci U.S.A 86: 1583-1587; a type I transglutaminase promoter (U.S. Pat. No. 5,643,746); and an involucrin promoter (Phillips et al. (2000) Biochem J 348:45-53). Expression may also be driven by a promoter of a housekeeping enzyme, e.g., EF1-α promoter, ribosomal protein L4 promoter, or phosphoglycerate kinase promoter.

Transgene expression can be driven by a more widely expressed cellular (e.g., GAPDH or other “housekeeping gene”) or even viral (e.g., CMV, HSV, etc.) promoter or promoter/enhancer combination. Experiments in mice have shown that the expression of transgenes from viral promoters in grafted keratinocytes tends to diminish over time, while the expression of transgenes driven by keratinocyte-specific promoters tends to be maintained in the graft. Therefore, it may be preferable to use keratinocyte-specific promoters to drive expression of the transgene. It is also known in the art that enhancers and promoters most often act as cassettes, such that the activity of a given promoter may be enhanced by an enhancer associated with a different gene than that with which the promoter is normally associated. The promoter may be inducible, by for example, a drug given either topically or systemically (e.g., tetracycline), or by a physical treatment (e.g., UV irradiation). Examples of such inducible promoters are disclosed, e.g., in U.S. Pat. No. 5,851,796 and U.S. Pat. No. 6,133,027. The selection of regulatory elements appropriate and functional for the expression of a given therapeutic transgene in LL, MLL or VLL cells is within the knowledge of one skilled in the art.

Selectable Markers

A variety of selectable markers can be incorporated into the LL, MLL and VLL keratinocytes. For example, a selectable marker which confers a selectable phenotype such as drug resistance, nutritional auxotrophy, resistance to a cytotoxic agent or expression of a surface protein, can be used. Selectable marker genes which can be used include neo, gpt, dhfr, ada, pac (puromycin), hyg and hisD. The selectable phenotype conferred makes it possible to identify and isolate recipient primary or secondary cells.

DNA Constructs

DNA constructs, which include exogenous DNA and, optionally, DNA encoding a selectable marker, along with additional sequences necessary for expression of the exogenous DNA in recipient LL, MLL or VLL cells can be used to genetically modify the recipient cells in which the encoded protein is to be produced. In other embodiments, infectious vectors, such as retroviral, herpes, lentivirus, adenovirus, adenovirus-associated, mumps and poliovirus vectors, can be used for this purpose.

A DNA construct which includes the exogenous DNA and additional sequences, such as sequences necessary for expression of the exogenous DNA, e.g., a promoter, can be used. A second DNA construct which includes DNA encoding a selectable marker, along with additional sequences, such as a promoter, polyadenylation site and splice junctions, can be used to confer a selectable phenotype upon introduction into LL, MLL or VLL keratinocytes. The two DNA constructs are introduced into LL, MLL or VLL keratinocytes, using methods described herein.

In other cases, one DNA construct which includes exogenous DNA, a selectable marker gene and additional sequences (e.g., those necessary for expression of the exogenous DNA and for expression of the selectable marker gene) can be used.

Transfection of LL, MLL or VLL Epithelial Cells

The cells described herein, e.g., the LL, MLL or VLL keratinocytes, can be combined with exogenous DNA to be stably integrated into their genomes and, optionally, DNA encoding a selectable marker, and treated in order to accomplish transfection. The exogenous DNA and selectable marker-encoding DNA can each be on a separate construct or on a single construct. An appropriate quantity of DNA to ensure that at least one stably transfected cell containing and appropriately expressing exogenous DNA is produced is used. In general, 0.1 to 500 μg DNA is used.

LL, MLL or VLL cells described herein, e.g., LL, MLL or VLL keratinocytes, can be transfected by electroporation. Electroporation is carried out at appropriate voltage and capacitance (and time constant) to result in entry of the DNA construct(s) into the LL, MLL or VLL keratinocytes. Electroporation can be carried out over a wide range of voltages (e.g., 50 to 2000 volts) and capacitance values (e.g., 60-300 μFarads). Total DNA of approximately 0.1 to 500 μg can be used.

LL, MLL or VLL cells can also be transfected using microinjection. Other known methods such as calcium phosphate precipitation, modified calcium phosphate precipitation and polybrene precipitation, liposome fusion and receptor-mediated gene delivery, and others, can be used to transfect cells. A stably, transfected cell is isolated and cultured and subcultivated, under culturing conditions and for sufficient time, to propagate the stably transfected cells and produce a clonal cell strain of transfected cells. More than one transfected cell can be cultured and subculturated, resulting in production of a heterogenous cell strain.

The transfected LL, MLL or VLL cells can be used to provide a therapeutic protein to an individual in effective amounts. The therapeutic protein can be isolated from the transfected cells or their culture media and administered to the individual. In some cases, the transfected cells are implanted or grafted into the individual and allowed to produce the therapeutic protein in vivo. The number of required cells for implantation of a transfected clonal or heterogenous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

Episomal Expression of Exogenous DNA

DNA sequences that are present within the cell yet do not integrate into the genome are referred to as episomes. Recombinant episomes may be useful in at least four settings: 1) if a given cell type is incapable of stably integrating the exogenous DNA; 2) if a given cell type is adversely affected by the integration of DNA; 3) if a given cell type is capable of improved therapeutic function with an episomal rather than integrated DNA; and 4) if the chromosomal integration of the exogenous DNA is undesirable.

Using transfection and culturing as described herein, exogenous DNA in the form of episomes can be introduced into the LL, MLL or VLL cells described herein, e.g., LL, MLL or VLL keratinocytes. Plasmids can be converted into such an episome by the addition of DNA sequences for the Epstein-Barr virus origin of replication and nuclear antigen (Yates (1985) Nature 319:780-7883). Vertebrate autonomously replicating sequences can be introduced into the construct (Weidle (1988) Gene 73:427-437). These and other episomally derived sequences can also be included in DNA constructs without selectable markers, such as pXGH5 (Selden et al. (1986) Mol Cell Biol 6:3173-3179). The episomal exogenous DNA can then be introduced into LL, MLL or VLL keratinocytes as described in this application (if a selective marker is included in the episome a selective agent is used to treat the transfected cells).

Implantation of Transfected LL, MLL or VLL cells

The genetically modified cells (or clonal or heterogenous cell strains) produced as described above can be introduced into an individual to whom the therapeutic protein is to be delivered, using known methods, using various routes of administration and at various sites (e.g., renal subcapsular, subcutaneous, central nervous system (including intrathecal), intravascular, intrahepatic, intrasplanchnic, intraperitoneal (including intraomental), or intramuscular implantation). In a preferred embodiment, upon the isolation of LL, MLL or VLL cells that stably carry a desired transgene, such cells can then be expanded in culture under conditions permitting the production of sheets of cells useful for tissue grafts as described above. Following expansion and establishment of transplantable cell sheets or matrices including LL, MLL or VLL cells, the cells can be transferred to a patient graft site prepared by removal of the epidermis. LL, MLL or VLL epithelial cells modified to produce a therapeutic gene product can be grafted under a flap of epidermis as taught by Gerrard et al. (1993) Nature Genetics 3: 180-183.

In one aspect, the LL, MLL or VLL cells described herein can be contained within a biocompatible matrix for implantation into a subject. For example, the cells can be contained within a matrix material that includes insoluble collagen fibrils. In addition, the cells can be contained in a matrix having microspheres added to a collagen matrix, thereby forming what is herein termed a “hybrid matrix” (e.g., a hybrid matrix as described in U.S. Pat. No. 5,965,125, which is incorporated herein by reference). Examples of microspheres which are described as consisting essentially of purified collagen include ICN Cellagen™. Beads and Cellex Biosciences macroporous microspheres. The microspheres are preferably of a porous consistency, but may be smooth, and typically have an approximately spherical shape with a diameter of approximately 0.1 to 2 mm (e.g., between approximately 0.3 and 1 mm).

A hybrid matrix can be formed by mixing microspheres with the LL, MLL or VLL cells (preferably LL, MLL or VLL cells that include an exogenous nucleic acid that causes the production of a therapeutic protein), and soluble collagen prior to gelling of the collagen to form the matrix. If desired, the microspheres and cells can be cultured together for a period which permits the cells to adhere to the microspheres before addition of the non-gelled collagen solution. Alternatively, the three constituents can be mixed essentially simultaneously or in any desired order, followed by gelation of the soluble collagen within the mixture, to form a gelled mixture consisting of insoluble collagen fibrils, cells and microspheres. This gelled mixture gradually becomes smaller through the exclusion of liquid to form a solid, relatively resilient, implantable unit that contains both the microspheres and the cells embedded in the insoluble collagen fibril network. When the microspheres are also composed largely of collagen, the resulting matrix is herein termed a “hybrid collagen matrix.”

The implantable matrices described herein, and further in U.S. Pat. No. 5,965,125, are useful for the administration of LL, MLL, or VLL cells described herein to a subject (preferably for the administration of LL, MLL or VLL cells expressing a therapeutic protein).

Uses for Genetically Modified LL, MLL or VLL Cells

Genetically modified LL, MLL or VLL cells have wide applicability as a factory, vehicle or delivery system for therapeutic proteins, such as enzymes, hormones, cytokines, antigens, antibodies, clotting factors, anti-sense RNA, regulatory proteins, transcription proteins, receptors, structural proteins, novel proteins and nucleic acid products, and engineered DNA that causes or alters the production of such proteins and other gene products, e.g., RNA. For example, an individual deficient in a particular enzyme is a candidate for enzyme replacement therapy with enzyme produced in vitro from the genetically modified LL, MLL or VLL cells described herein. An individual deficient in a particular enzyme can also be provided the replacement enzyme by implantation of the genetically modified LL, MLL or VLL cells described herein, such that the enzyme is produced in vivo from the implanted, genetically modified cells.

For example, an individual who has been diagnosed with Hemophilia A, a bleeding disorder that is caused by a deficiency in Factor VIII, a protein normally found in the blood, can be provided Factor VIII produced in vitro or in vivo from the cells of the invention. In another example, an individual who has been diagnosed with Hemophilia B, a bleeding disorder that is caused by a deficiency in Factor IX, a protein normally found in the blood, can be provided Factor IX produced in vitro or in vivo from the cells of the invention. A similar approach can be used to treat other conditions or diseases. For example, short stature can be treated by administering human growth hormone (hGH) produced in vitro or in vivo from the genetically modified LL, MLL or VLL cells described herein; anemia can be treated by administering erythropoietin (EPO) produced in vitro or in vivo from the genetically modified LL, MLL or VLL cells described herein to an individual; diabetes can be treated by administering glucogen-like peptide-1 (GLP-1) produced in vitro or in vivo from GLP-1-expressing genetically modified LL, MLL or VLL cells described herein. A lysosomal storage disease (LSD) can also be treated by this approach. LSD's represent a group of at least 41 distinct genetic diseases, each one representing a deficiency of a particular protein that is involved in lysosomal biogenesis. A particular LSD can be treated by providing a lysosomal enzyme produced in vitro or in vivo from genetically modified LL, MLL or VLL cells that express the lysosomal enzyme. Fabry Disease can be treated by administering α-galactosidase produced in vitro or in vivo from α-galactosidase-expressing LL, MLL or VLL cells; Gaucher disease can be treated by administering glucocerebrosidase produced in vitro or in vivo from glucocerebrosidase-expressing genetically modified LL, MLL or VLL cells; MPS (mucopolysaccharidosis) type I (Hurler-Scheie syndrome) can be treated by administering α-L-iduronidase produced in vitro or in vivo from α-L-iduronidase-expressing genetically modified LL, MLL or VLL cells; MPS type II (Hunter syndrome) can be treated by administering iduronate-2-sulfatase produced in vitro or in vivo from iduronate-2-sulfatase-expressing genetically modified LL, MLL or VLL cells; MPS type III-A (Sanfilipo A syndrome) can be treated by administering Heparan N-sulfatase produced in vitro or in vivo from Heparan N-sulfatase-expressing genetically modified LL, MLL or VLL cells; MPS type III-B (Sanfilipo B syndrome) can be treated by administering α-N-acetylglucosaminidase produced in vitro or in vivo from α-N-acetylglucosaminidase-expressing genetically modified LL, MLL or VLL cells; MPS type III-C (Sanfilipo C syndrome) can be treated by administering acetyl coenzyme A:α-glucosaminide acetyltransferase produced in vitro or in vivo from acetyl coenzyme A:α-glucosaminide acetyltransferase-expressing genetically modified LL, MLL or VLL cells; MPS type III-D (Sanfilippo D syndrome) can be treated by administering N-acetylglucosamine-6-sulfatase produced in vitro or in vivo from N-acetylglucosamine-6-sulfatase-expressing genetically modified LL, MLL or VLL cells; MPS type IV-A (Morquio A syndrome) can be treated by administering galactose-6-sulfatase produced in vitro or in vivo from galactose-6-sulfatase-expressing genetically modified LL, MLL or VLL cells; MPS type IV-B (Morquio B syndrome) can be treated by administering β-galactosidase produced in vitro or in vivo from β-galactosidase-expressing genetically modified LL, MLL or VLL cells; MPS type VI (Maroteaux-Lamy syndrome) can be treated by administering N-acetylgalactosamine-4-sulfatase (Arylsulfatase B) produced in vitro or in vivo from N-acetylgalactosamine-4-sulfatase (Arylsulfatase B)-expressing genetically modified LL, MLL or VLL cells; MPS type VII (Sly syndrome) can be treated by administering β-glucuronidase produced in vitro or in vivo from β-glucuronidase-expressing genetically modified LL, MLL or VLL cells.

Administration

A patient in need of a therapeutic protein, e.g., a therapeutic enzyme, can be treated by introducing into the patient a therapeutically effective amount of purified protein, preferably a human protein, obtained from cultured LL, MLL or VLL cells genetically modified to express, and optionally secrete, the protein. The purified protein can be administered to a subject by standard methods. For example, the agent can be administered by any of a number of different routes including intravenous, intradermal, subcutaneous, oral (e.g., inhalation), transdermal (topical), transmucosal, or as a solid implant.

The purified protein can be incorporated into pharmaceutical compositions suitable for administration to a subject, e.g., a human. Such compositions typically include the protein and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the therapeutic protein in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Genetically modified LL, MLL or VLL cells can be administered in a number sufficient to ameliorate the symptoms of the disease or disorder being treated. The number of cells sufficient to ameliorate the symptoms of a disease or disorder will vary depending upon the nature of the disease, the level of expression and/or secretion of the gene product, and upon the efficiency with which the gene product is delivered to the circulation. The practitioner administering the cells can determine the number of cells necessary to ameliorate the symptoms of the disease or disorder being treated, and can monitor the symptoms as a measure of the success of treatment. In addition to monitoring symptoms, the serum levels of a therapeutic polypeptide can be measured using an immunoassay or, if the polypeptide is an enzyme, a direct assay for enzyme activity. The number of cells will vary not only with the disease being treated, but also with the level of expression of a therapeutic gene in a given cell clone. Also, doses of cells will vary with the efficiency with which a given therapeutic gene product is released to the circulation.

The number of cells administered can range from about 10⁶ to about 10¹⁰ cells, most often from about 10⁶ to about 10⁹ cells.

One may determine the success of cell implantation or grafting using LL, MLL or VLL cells by analyzing a biopsy of the graft or implantation site after a given amount of time. In addition, when the implanted cells express a transgene, the biopsy can be examined for the expression of the transgene by, for example, immunohistochemical means or RT-PCR. The expression of a transgene introduced to the LL, MLL or VLL cells can persist at the graft site. A graft or implant of transfected cells is considered successful if transgene expression is detectable through immunohistochemistry, RT-PCR, or other means after 2 weeks, 3 months or longer, preferably a year or more, and more preferably 5 years or more, a decade or more, or even the natural lifespan of the recipient.

The LL, MLL or VLL cells used for gene therapy can be patient-specific genetically-engineered cells. It is possible, however, to obtain cells from another individual of the same species or from a different species. Use of such cells might require administration of an immunosuppressant, alteration of histocompatibility antigens, or use of a barrier device to prevent rejection of the implanted cells.

When transfected LL, MLL or VLL keratinocytes are used, the need for multiple grafts throughout the lifetime of the graft recipient is reduced because the human LL, MLL or VLL epithelial cells have sufficient proliferative potential to maintain a graft over a long period of time, preferably as long as the lifetime of a graft recipient. Because of this, in some cases a one-time grafting treatment with LL, MLL or VLL cells will be sufficient. For some, multiple grafts with non-LL, MLL or VLL cells can be necessary until a LL, MLL or VLL cell can be identified and/or isolated for treatment.

In Vitro Assays

LL, MLL or VLL cells, e.g., the LL, MLL or VLL keratinocytes described herein, can be used in in vitro assays designed to evaluate drugs, or generally for treatments affecting the skin. For example, drugs or other treatments can be evaluated, e.g., for toxicity to or tendency to transform keratinocytes. Such evaluations can be made, e.g., by adding the composition to the culture medium of cells cultured under proliferative conditions as described herein, by adding the composition to cells in organotypic cultures, or by adding the composition to a graft of the LL, MLL or VLL cells described herein, e.g., in an animal. LL, MLL or VLL cells can be evaluated for toxicity caused by a given agent or treatment by morphological criteria and by vital assays using standard methods known in the art (e.g., trypan blue dye exclusion or the MTT assay). Cells can be evaluated for transformation by morphological criteria, culture in semi-solid medium (soft agar assays) and by tumor formation assays in nude mice. Cells can also be evaluated for loss of the ability to differentiate by monitoring expression of differentiation markers of cells treated with a given agent relative to cells that have not been treated.

The effects of chemotherapeutic agents on normal epidermal keratinocytes can be evaluated using LL, MLL or VLL cells. This may be performed by e.g., contacting the LL, MLL or VLL cells with the agent being tested and monitoring growth, differentiation or cell death in those cultures. LL, MLL or VLL cells can be co-cultured with tumor cells, such as squamous cell carcinoma cells, in order to more closely simulate the biology of tumors in vivo. Tumor cells and normal cells exist in close proximity in vivo, and the cells influence each other by, for example, secretion of growth factors or by causing local ischemia. Antitumor agents or treatments can be screened for efficacy (i.e., cytotoxic or cytostatic effect) against tumor cells in the presence of normal cells using co-culture of LL, MLL or VLL cells and a tumor cell line. Co-culture in an in vitro model of a stratified squamous cell epithelium can be used. In either case, the effects of candidate antitumor agents on one or both the tumor cells and the LL, MLL or VLL cells can be evaluated, in order to identify those agents that are effective against the tumor cells but do not kill or severely impair the functions of the normal LL, MLL or VLL cells. An organotypic co-culture system that simulates a stratified squamous cell epithelium has the advantage of more closely reproducing in vivo the tumor cell microenvironment. Co-culture systems can be used to screen for antitumor activity of novel drugs or treatments, as well as to evaluate the effects of novel combinations of known drugs or treatments. For example, the ability of a known drug to render tumor cells susceptible to another drug or treatment, such as irradiation, may be evaluated.

An organotypic culture system that reproduces the architecture of a stratified squamous cell epithelium can be established by seeding LL, MLL or VLL cells onto a collagen layer containing normal human fibroblasts (isolated, for example, from a skin biopsy). Organotypic culture systems simulating human skin are described by, for example, Javaherian et al. (1998) Cancer Res. 58: 2200-2208, and Garlick & Taichman (1994) Lab. Invest 70: 916-924. Javaherian et al., in particular, describes co-culture of transformed and normal human keratinocytes under conditions that simulate the tumor cell microenvironment.

In addition to screening assays for the identification of novel drugs or novel combinations of existing drugs, LL, MLL or VLL cells may be used to evaluate the effects of known drugs or treatments on tumors in a patient-specific manner in order to tailor a therapeutic regimen. In this case, cells from a patient's tumor would be used instead of cells from a tumor cell line.

In any of the co-culture-based uses of LL, MLL or VLL cells, the LL, MLL or VLL cells or the tumor cells may be tagged by expression of a detectable marker, such as green fluorescent protein (GFP), in order to differentiate them from one another. Methods of introducing a tag expression vector are described herein and/or known in the art.

All patents and other publications cited herein are incorporated herein by reference in their entirety.

A number of embodiments of the invention have been described. Nevertheless, it will be understood that various modifications may be made without departing from the spirit and scope of the invention. Accordingly, other embodiments are within the scope of the claims.

### EXAMPLES

### Example 1

### Impact Of CFE And PDT On Population Drift

This example illustrates that growth rate or population doubling time (PDT) and colony forming efficiency (CFE) can lead to population drift.

Cumulative cell output (CCO) is defined as (cell output−cell input)/(fraction of total used as input). CCO is related to CFE and PDT by the following equation: CCO=(CFE)ⁿ (2^{(t)/(PDT)}), where CFE is colony forming efficiency; n is the number of passages; t is the time elapsed; and PDT is the population doubling time.

Table 1 illustrates the impact of CFE on two clones, A and B, initially represented at a 1:1 ratio. Changes in the representation will be equal to (CFE_{A}/CFE_{B})ⁿ. Table 2 illustrates the impact of PDT on cell output.

TABLE 1
Impact of CFE on cell output

| | clone A: clone B | clone A: clone B | clone A: clone B | clone A: clone B |
|---|---|---|---|---|
| | after pass 2 | after pass 7 | after pass 14 | after pass 21 |
| CFE_{A}/CFE_{B} | (˜14 generations*) | (˜50 generations*) | (˜100 generations*) | (˜150 generations*) |
| 1.1 | 1.21 | 1.9 | 3.8 | 7.4 |
| 1.2 | 1.44 | 3.5 | 12.8 | 98.9 |
| 1.5 | 2.25 | 17.0 | 292.0 | 4.9 × 10³ |
| 2.0 | 4.00 | 128.0 | 1.6 × 10⁴ | 2.1 × 10⁶ |

*assumes a 24 hour PDT and weekly passage of cultures

TABLE 2
Impact of PDT on cell output

| cell number | cell number | cell number | cell number | cell number |
|---|---|---|---|---|
| PDT (hours) | (14 days) | (50 days) | (100 days) | (150 days) |
| 24 | 1.6 × 10⁴ | 1.1 × 10¹⁵ | 1.3 × 10³⁰ | 1.4 × 10⁴⁴ |
| 22 | 4.0 × 10⁴ | 2.6 × 10¹⁶ | 6.9 × 10³² | 1.8 × 10⁴⁹ |
| 20 | 1.1 × 10⁵ | 1.1 × 10¹⁸ | 1.3 × 10³⁶ | 1.5 × 10⁵⁴ |
| 18 | 4.2 × 10⁵ | 1.2 × 10²⁰ | 1.4 × 10⁴⁰ | 1.6 × 10⁶⁰ |

Changes in representation of the two clones, A and B (Table 1), will be equal to: (2^{(t)/(PDT)}_{A})/(2^{(t)/(PDT)}_{B})=2^{(t)/(1/PDT}_{A}^{-1/PDT}_{B}). As shown in Table 2, even two-hour differences in doubling time can result in population drift. For example, if two clones are initially represented at a 1:1 ratio, clone A having a PDT of 18 hours and clone B having a PDT of 20 hours, clone A will take over the population. At 14 days A/B shifts from 1 to 3.8; at 50 days, A/B is 109.1; at 100 days A/B is 1.1×10⁴; and at 150 days A/B is 1.1×10⁶. Therefore, isolating cells after multiple passages and numerous population doublings ensures that a proportion of clones will be missed, some of which may have great proliferative potential.

### Example 2

### Analyses of Very Long Lived Epidermal Cell Clones

Keratinocyte clones isolated directly from primary cell cultures, as described herein, have been examined to determine any possible relationship between proliferative capacity and CFE or clonal type.

A. Determination of the Longevity of Clones

The proliferative potential of keratinocyte clones isolated from biopsies of three males and five females was determined by performing serial passaging. In this experiment, keratinocytes were passaged in mass culture until their proliferative potential had been exhausted or until they had undergone 300 population doublings. Culture senescence was defined as the point at which two successive colony forming efficiencies (CFE's) were below 1. At each passage, CFE was determined. At the second passage (approximately generation 6), between 6 and 28% of clones terminated (Table 4). Clones that underwent more than 100 doublings were considered long-lived (LL). LL clones were described by the present inventor in Matic et al. (1999) Journal of Investigative Dermatology 112(4):622;595a. In the data described herein, two groups of LL cells have been identified and characterized. The first group consists of moderately long-lived (MLL) clones. The MLL clones underwent between 100 and 200 doublings prior to senescence. Approximately 0-8.6% of the clones from three males and five females were MLL. In contrast to the MLL clones, which entered senescence during the course of the study, very long-lived (VLL) clones did not exhaust their proliferative potential during the course of the experiments shown (>350 doublings). Between 6.1-8.2% of the clones from each male donor were VLL, and 0-7.4% of the clones from each female donor were VLL. Female donor 2 had no MLL or VLL clones. There was no correlation between the percentage of clones that died at passage 2 and the percent of LL clones. Likewise, the generation number at which half of the clones terminated was not indicative of either the percent of LL clones or the culture lifespan (Table 4).

Cultures can consist of heterogeneous colony types. As expected, cultures of senescing clones had a high percent of terminal (abortive) colonies. However, even in these cultures, colonies could be found that outlived the parent clone. In one experiment sub-cloning was carried out on the three remaining colonies from the final mass culture of a clone of female donor 1. Two of the colonies were irregularly shaped and had large cells. The cells from these colonies could not be subcultured further. The one colony that contained small cells was passaged more than 50 times and thus could be classified as VLL.

B. No Correlation Between CFE and Proliferative Potential

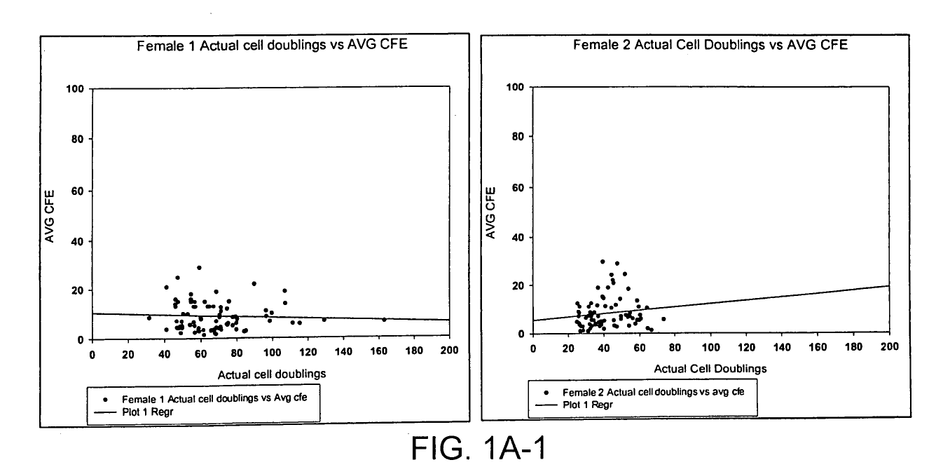

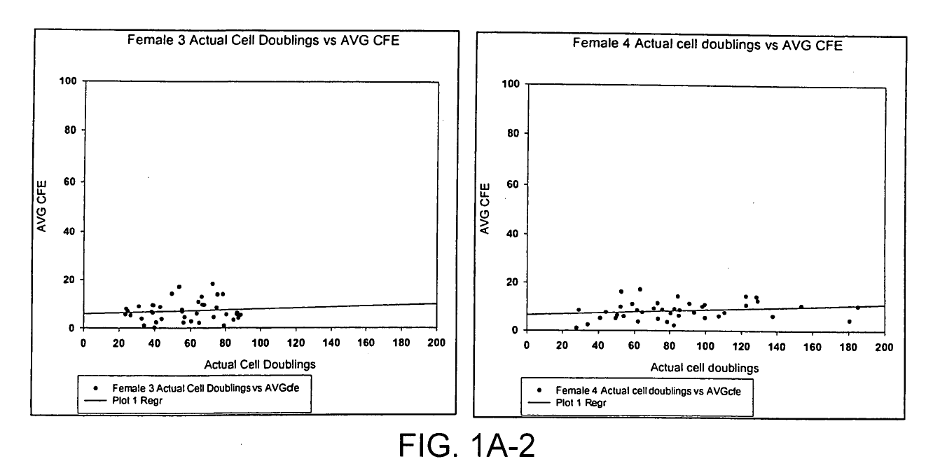

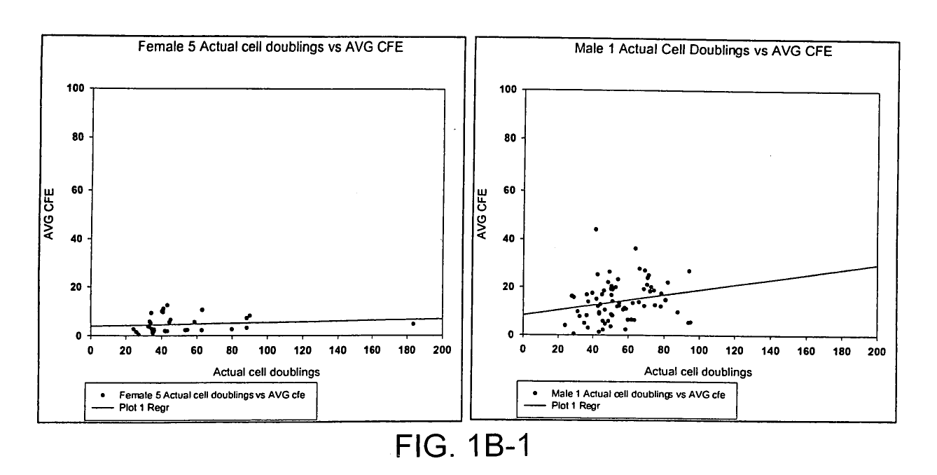

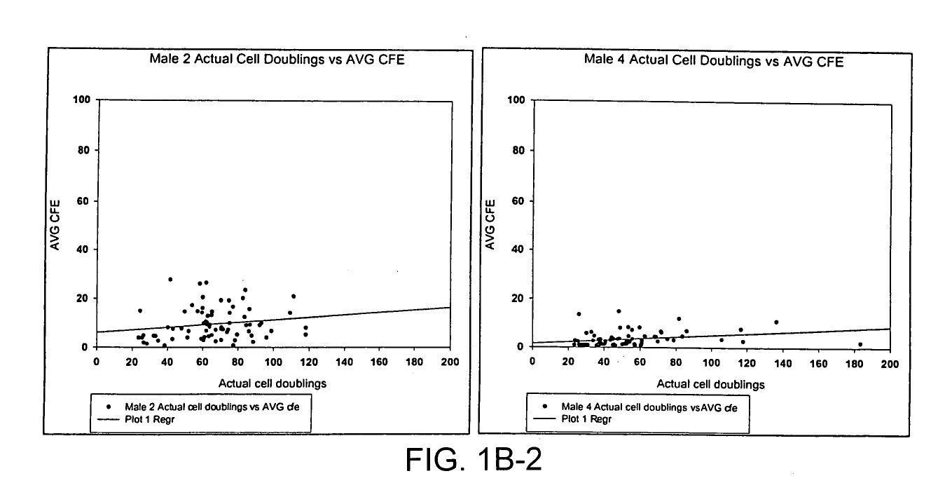

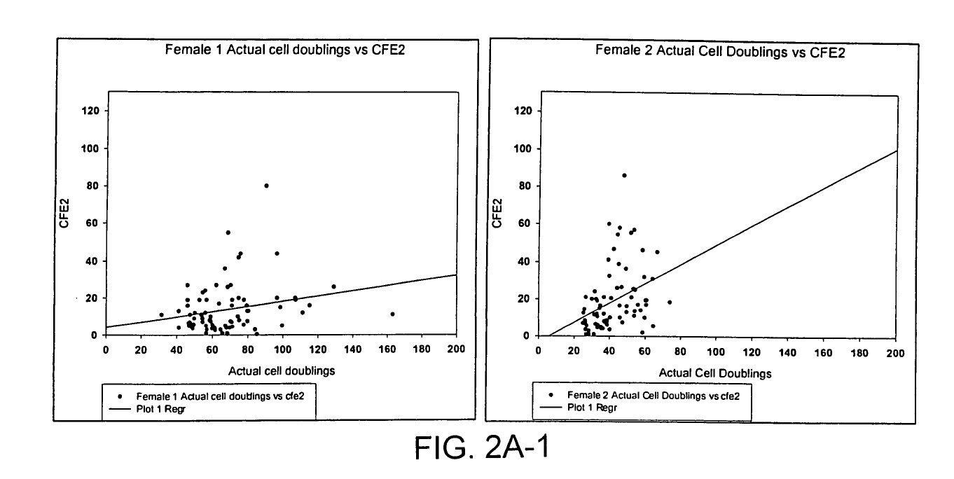

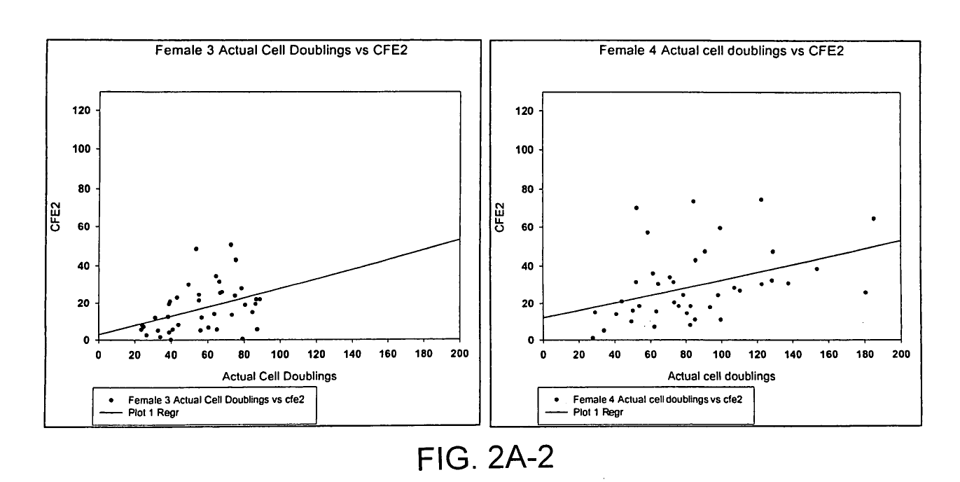

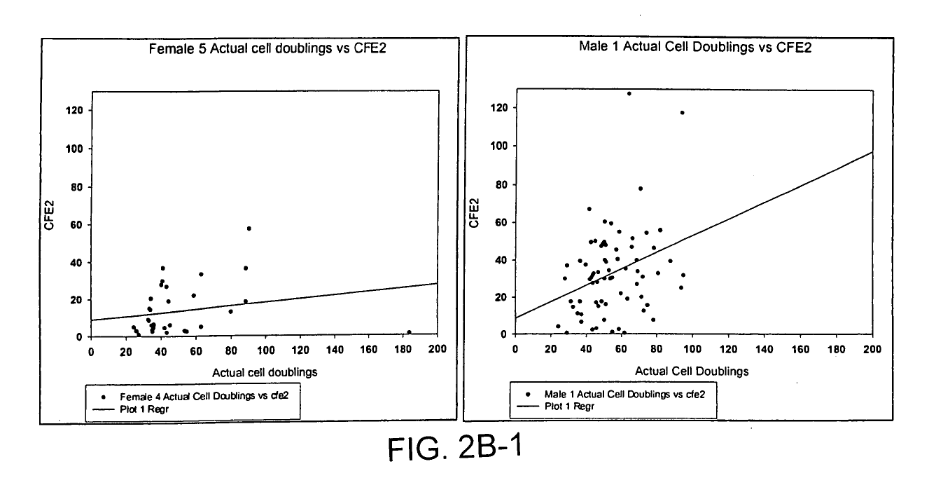

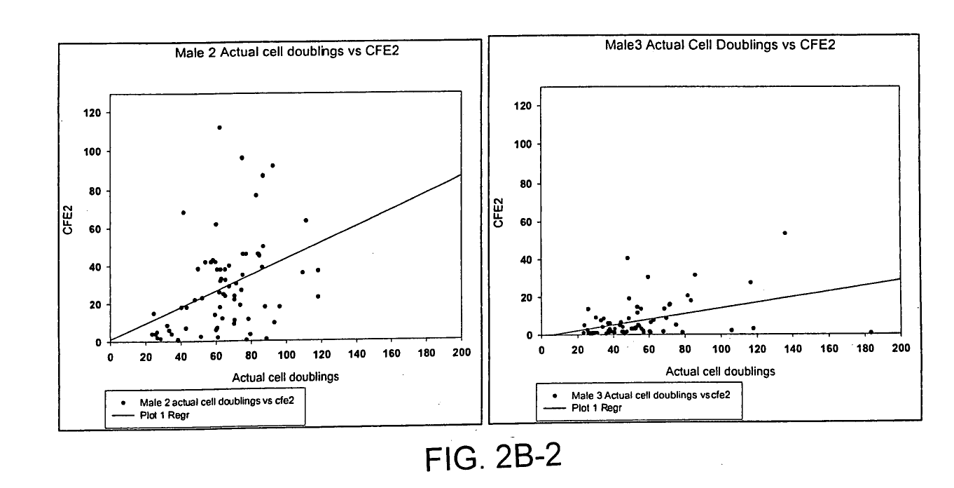

It has often been suggested that CFE is indicative of longevity in culture. This was re-evaluated by comparing the average CFE (CFEₐᵥₑ; FIG. 1) or the CFE of the clones at the second passage (CFE2; FIG. 2) with culture life span. VLL clones that did not enter senescence within 300 doublings were not included in these analyses. As seen in FIGS. 1 and 2 (see Table 3 for corresponding P values) there was no statistically significant correlation between CFE and longevity in culture.

As the number of cells derived from any single cell is a function of both CFE and PDT, cells with the shortest PDT and greatest CFE will overtake the population prior to senescence (see Example 1). Because this phenomenon is independent of proliferative potential, early cell cloning is a requirement for preventing unintentional loss of LL. MLL or VLL cells that may have lower CFE or PDT.

TABLE 3
Statistical Analysis of Data in FIGS. 1A-B and 2A-B

| | P values | | | |
|---|---|---|---|---|
| | Actual- | | Correlation values | |
| Donors | cfe2 | Actual-avg cfe | Actual-cfe2 | Actual-avg cfe |
| Male 1 | 1.70E−09 | 0.00 | −0.003701403 | 0.442715141 |
| Male 2 | 0.00 | 0.00 | 0.509752559 | 0.494269887 |
| Male 3 | 0.00 | 0.00 | 0.21954345 | 0.645295727 |
| Female 1 | 0.00 | 0.00 | 0.166126181 | −0.080757299 |
| Female 2 | 0.00 | 0.00 | 0.342769073 | 0.150455126 |
| Female 3 | 0.00 | 0.00 | 0.393231172 | 0.09792315 |
| Female 4 | 0.00 | 0.00 | 0.195908978 | 0.595594554 |
| Female 5 | 6.0E−08 | 3.00E−10 | 0.327410298 | 0.590404507 |

C. Clonal Analyses

Earlier work performed with foreskin keratinocytes suggested that the percentage of terminal colonies within a clone (the colonies smaller than 2-5 mm), could be used to predict longevity of the clone (Barrandon & Green (1987) Proc Natl Acad Sci USA 84: 2302-2306). Barrandon & Green distinguished three clonal types for epidermal keratinocytes based upon the frequency of terminal colonies (colonies <5 mm²). A clone was classified as a paraclone when all colonies formed were terminal or when no colonies formed. When more than 5% but less than 95% of the colonies were terminal, the clone was classified as a meroclone. The clones with fewer than 5% terminal colonies were classified as holoclones. It has been often assumed that stem cells in vitro that exhibit holoclone characteristics have high colony forming efficiency (CFE), and have growth potentials that exceed those of other keratinocytes. Surprisingly, however, as is demonstrated herein below, there is no apparent correlation between the holoclone type or CFE and the proliferative potential of the cells.

Long lived (LL) clones, clones that underwent more than 100 population doublings, were analyzed for the presence of terminal colonies (Table 5). Colonies smaller than 2 mm, but with smooth edges and small cells were identified as satellite colonies (Randolph & Simon (1993) J Biol Chem 268: 9198-9205), and were not classified as terminal. Only 12 out of 53 LL clones (6/29 VLL and 6/24 MLL clones) could be classified as holoclones. Male donor 2 had the highest percentage of holoclones within the long-lived population, 60% (3 VLL and 3 MLL clones). This donor's clones were the only ones where a correlation, albeit weak, was found between CFEs and longevity in culture. Two out of 92 non-LL clones from this donor were holoclones. No holoclones were isolated from male donor 3 and female donors 2, 3 and 4. When all clones were considered (rather than just LL clones), a weak negative correlation was found but it was not statistically significant.

D. Analysis of Long Lived Clones by Cumulative Cell Output

It is estimated, based on a life span of 100 years and a complete epidermal turnover rate of once every 4 weeks, that about 5.72×10⁹ cells would be needed to replenish a 1 cm² region of tissue over a lifetime. CCO was determined for clones isolated directly from primary cultures in order to measure whether keratinocytes isolated from adult human epidermis have sufficient replicative potential to maintain a graft over a lifetime. As seen in Table 6, relatively high percentages (21.7-70.4%) of the clones demonstrated sufficient potential to perform this task (this number excludes those clones that did not undergo senescence during the course of the experiments, i.e., the VLL clones). Female donor 2, whose cells in general had a poorer potential in vitro compared to other donors, had the lowest percentage (21.7%) of clones that were able to regenerate 1 cm² of epidermis for 100 years, but nonetheless yielded cells capable of the task. The largest variation was observed in male donor 2, which gave CCOs that ranged from 4.9×10¹⁸-9.6×10⁹⁰ cells. The smallest CCO was generated by clones of female donor 2, 5.1×10¹⁰-7.5×10¹². As seen in Table 6, if one calculates the proliferative potential based upon the number of generations prior to senescence rather than by CCO, a significantly higher percentage of clones are capable of maintaining a 1 cm² epidermis for 100 years (55.3%-86%). This calculation excludes the effects of cell passage.

It has been suggested that one of the ways to identify stem cells is to assess their in vitro proliferative potential and growth characteristics. The growth characteristics, which have been assumed to correlate with proliferative potential include CFE and clonal type (holoclone, meroclone, paraclone). However, neither characteristic showed significant correlation with in vitro proliferative potential. The lack of correlation observed between CFE2 or CFEₐᵥₑ and proliferative potential is in accordance with recently reported data (Li, et al. (1998) Proc Natl Acad Sci USA 95: 3902-3907). More surprising were the results on clonal type. The absence of the holoclones did not predict limited proliferative potential. Only one out of eight donors had a high percentage (42-60%) of holoclones in the LL population. Four out of eight donors had no holoclones. The discrepancy between these results and those of the initial report of Barrandon and Green (supra; however, see Rochat et al. (1994) Cell 76: 1063-1073) may be due to differences between donors, culture conditions, or donor body site and age (the previous observations were made using keratinocytes from neonatal foreskin)

Excluding VLLs, about 22%-70% (based on CCO) and 55%-86% (based on number of generations prior to senescence) of top 10% longest living clonogenic cells, most of which were meroclones, had sufficient proliferative potential to meet the criteria for stem cells that are currently widely accepted in the field (Lajtha, supra). 7.7% of 649 clones analyzed had an exceptionally high proliferative potential.

The data presented herein indicate that the stem cell or stem cell-like pool may consist of cells with different proliferative potentials. The different proliferative potentials of stem cells may reflect their tissue history, i.e. the number of generations a particular cell underwent prior to its isolation from the tissue. In fact, stem cell hierarchy may be viewed as a part of the strict regulatory system that controls stem cell divisions.

TABLE 4
Proliferative potential of clonal isolates

| | | | % Clones | | | |
|---|---|---|---|---|---|---|
| | Number of | % Clones | terminating | | | Generation at |
| | Colonies | terminating | between | % MLL | % VLL | which ½ |
| Donor | Cloned | by gen. 6 | gen. 7-99 | Clones | Clones | clones senesced |
| Male 1 | 84 | 6.0 | 84.6 | 0 | 7.1 | 51 |
| Male 2 | 98 | 15.3 | 72.4 | 4.1 | 8.2 | 63 |
| Male 3 | 114 | 28.0 | 60.6 | 5.3 | 6.1 | 37 |
| Female 1 | 93 | 11.8 | 79.5 | 8.6 | 1.1 | 63 |
| Female 2 | 106 | 20.8 | 74.5 | 0 | 0 | 33 |
| Female 3 | 50 | 10.0 | 78.0 | 0 | 2.0 | 44 |
| Female 4 | 54 | 14.8 | 76.0 | 1.9 | 7.4 | 76 |
| Female 5 | 50 | 26.0 | 65.8 | 2.1 | 6.1 | 35 |

There were no statistically significant differences between males and females.

TABLE 5
Correlation between proliferative potential and clonal phenotype.

| | | | | | |
|---|---|---|---|---|---|
| | | | Number of | Number of | |
| | | Number of | Terminal | Satellite | % |
| Clone # | Clone # | Colonies | Colonies | Colonies | Terminal |
| (VLL) | (MLL) | >5 mm² | (<5 mm²) | (<5 mm²) | Colonies |

A: Male 1

| | | | | |
|---|---|---|---|---|
| 25 | 94 | 5 | 1 | 5* |
| 29 | 26 | 12 | 1 | 36 |
| 34 | 62 | 22 | 3 | 31 |
| 57 | 19 | 24 | 0 | 56 |
| 64 | 50 | 3 | 7 | 5* |
| 78 | 80 | 9 | 2 | 10 |

B: Male 2

| | | | | | |
|---|---|---|---|---|---|
| 24 | | 98 | 4 | 4 | 4* |
| 36 | | 138 | 2 | 0 | 1* |
| 44 | | 118 | 9 | 0 | 6 |
| 49 | | 52 | 4 | 0 | 7 |
| 56 | | 170 | 7 | 0 | 4* |
| 67 | | 180 | 24 | 0 | 12 |
| 84 | | 234 | 21 | 0 | 8 |
| | 3 | 68 | 3 | 0 | 4* |
| | 30 | 126 | 6 | 4 | 4* |
| | 58 | 74 | 4 | 0 | 5* |

C: Male 3

| | | | | | |
|---|---|---|---|---|---|
| 36 | | 17 | 11 | 0 | 36 |
| 39 | | 21 | 3 | 0 | 13 |
| 79 | | 11 | 1 | 0 | 8 |
| 88 | | 41 | 12 | 0 | 23 |
| 93 | | 44 | 6 | 0 | 12 |
| 104 | | 20 | 36 | 0 | 64 |
| | 19 | 41 | 25 | 0 | 38 |
| | 42 | 1 | 3 | 0 | 75 |
| | 60 | 6 | 9 | 0 | 60 |
| | 98 | 107 | 13 | 0 | 11 |
| | 113 | 6 | 4 | 0 | 40 |

D: Female 1

| | | | | | |
|---|---|---|---|---|---|
| 4 | | 50 | 6 | 0 | 11 |
| | 9 | 21 | 1 | 0 | 5* |
| | 21 | 136 | 20 | 0 | 13 |
| | 24 | 28 | 20 | 0 | 42 |
| | 35 | 41 | 2 | 0 | 5* |
| | 43 | 88 | 9 | 0 | 9 |
| | 53 | 36 | 5 | 0 | 12 |
| | 83 | 104 | 4 | 0 | 4* |

E: Female 3

| | | | | |
|---|---|---|---|---|
| 7 | 13 | 10 | 0 | 43 |

F: Female 4

| | | | | | |
|---|---|---|---|---|---|
| 2 | | 65 | 24 | 2 | 26 |
| 4 | | 26 | 6 | 0 | 19 |
| 24 | | 7 | 16 | 0 | 70 |
| 41 | | 90 | 10 | 2 | 11 |
| 46 | | 54 | 8 | 0 | 13 |
| | 7 | 44 | 10 | 0 | 17 |
| | 13 | 45 | 14 | 0 | 24 |
| | 14 | 41 | 16 | 8 | 25 |
| | 16 | 53 | 16 | 1 | 23 |
| | 20 | 111 | 12 | 1 | 10 |
| | 21 | 53 | 6 | 0 | 10 |
| | 33 | 141 | 19 | 0 | 12 |
| | 43 | 65 | 22 | 0 | 25 |

G: Female 5

| | | | | | |
|---|---|---|---|---|---|
| 4 | | 30 | 1 | 0 | 3* |
| 5 | | 89 | 7 | 0 | 8 |
| 50 | | 38 | 9 | 0 | 24 |
| | 6 | 2 | 0 | 0 | NA |

Evaluation was carried out on all colonies of VLL and MLL clones from male 1 (A), male 2 (B), male 3 (C), female 1 (D), female 3 (E), female 4 (F), and female 5 (G). Female 2 had no LL clones. Any samples with 5% or fewer terminal colonies are defined as holoclones (*). Significant biopsy to biopsy variation was noted

TABLE 6
Clones capable of supporting epidermal homeostasis.

| | | | Percentage of clones capable | |
|---|---|---|---|---|
| | | | of maintaining | |
| | | | a 1 cm² epidermis for 100 | |
| | | | years | |
| | Potential cell output | | | Based on total |
| | of the 10% longest living clones | | Based on | cell counts at |
| | | Based on total cell | number of | all passages |
| | Based on | counts at all passages | generations | prior to |
| | number of generations | prior to senescence | prior to | senescence |
| Donor | prior to senescence | (CCO) | senescence | (CCO) |
| Male1 | 2.3 × 10²⁸-4.6 × 10¹⁰⁷ | 2.3 × 10¹⁴-2.0 × 10⁸⁰ | 85.7 | 47.6 |
| Male2 | 3.5 × 10³⁵-9.7 × 10¹¹⁷ | 4.9 × 10¹⁸-9.6 × 10⁹⁰ | 77.6 | 59.2 |
| Male3 | 3.3 × 10³⁵-2.1 × 10¹²⁶ | 2.8 × 10¹⁶-1.7 × 10⁷⁰ | 55.3 | 28.1 |
| Female1* | 1.0 × 10³⁰-2.6 × 10¹⁰⁸ | 7.9 × 10¹⁴-1.1 × 10⁴⁰ | 86.0* | 62.4* |
| Female2 | 4.5 × 10¹⁷-1.7 × 10²² | 5.1 × 10¹⁰-7.5 × 10¹² | 55.7 | 21.7 |
| Female3 | 2.8 × 10²⁵-5.2 × 10²⁶ | 3.6 × 10¹²-9.3 × 10¹⁴ | 68.0 | 36.0 |
| Female4 | 9.1 × 10⁶⁵-6.3 × 10¹⁰⁶ | 4.6 × 10³³-3.1 × 10⁷¹ | 79.6 | 70.4 |
| Female5 | 1.3 × 10²⁷-7.5 × 10⁷⁵ | 9.0 × 10¹³-2.1 × 10⁵⁹ | 64.0 | 20.0 |

*For this donor only there is no record of the number of clones which did not grow subsequent to passage 1. Therefore the % of clones capable of maintaining a 1 cm² epidermis for 100 years may be lower. For all other clones this number was recorded and was included in the calculations.
Note:
Differences between the cell output calculated based on number of generations and based on the total cells counted at all passages reflects the impact of CFE and passage number.

The epidermis contains 4.4×10⁶ cells/cm² and turns over approximately 13 times per year. Maintaining a viable 1 cm² epidermis for 100 years would then require approximately 5.7×10⁹ cells. This is approximately 2³³ or 33 generations.

E. Karyotype Analysis of MLL and VLL Clones

Karyotype analysis was performed on VLL clones isolated as described herein. Karyotype analysis was performed on one VLL clone at early passage (passage 4). The karyotype of the clone was normal.

Karyotype analysis was also performed on 26 VLL clones at late passage, from a total of 6 strains. Of these, one clone (a different clone than that karyotyped at early passage) had cells with a normal karyotype at passage 51. The others had one or more of several kinds of changes from normal, e.g., most clones had an iso8q. karyotype, and several had a change in the long arm of chromosome 20.

What is claimed is:

1. A method of producing a preparation of keratinocytes, said method comprising
providing a source of human epithelial tissue;
isolating at least one keratinocyte clone from the human epithelial tissue;
determining if the clone is capable of at least 150 population doublings after isolation from human tissue,
thereby providing said preparation.

2. The method of claim 1, wherein the human epithelial tissue is skin.

3. The method of claim 1, wherein the human epithelial tissue is adult tissue.

4. The method of claim 2, wherein the skin is adult skin.

5. The method of claim 1, wherein the keratinocyte clone is isolated prior to a time sufficient for seven doublings from the time the sample of human tissue is obtained.

6. The method of claim 1, wherein the keratinocyte clone is isolated prior to, or prior to a time sufficient for, four doublings from the time the sample of human tissue is obtained.

7. The method of claim 1, wherein the keratinocyte clone is isolated prior to, or prior to a time sufficient for, two doublings from the time the sample of human tissue is obtained.

8. The method of claim 1, wherein the keratinocyte clone is isolated directly from the human tissue.

9. The method of claim 1, wherein determining if the clone is capable of at least 150 population doublings after isolation from human tissue comprises providing a cell from the clone and performing a cell division assay on said cell.

10. The method of claim 1, wherein determining if the clone is capable of at least 150 population doublings after isolation from human tissue comprises:
dividing the clone into at least two aliquots;
performing serial passaging of the cells of one of the aliquots until the proliferative potential of said cells is exhausted or until said cells undergo 150 population doublings from the time of isolation from human tissue;
thereby determining if the clone is capable of at least 150 population doublings.

11. The method of claim 1, wherein the human tissue is skin, and
the keratinocyte clone is isolated prior to, or prior to a time sufficient for, four doublings from the time the sample of human tissue is obtained; and
determining if the clone is capable of at least 150 population doublings after isolation from human tissue comprises performing a cell division assay on said cell.

12. The method of claim 1, wherein the human tissue is skin, and
the keratinocyte clone is isolated directly from the human tissue, and
determining if the clone is capable of at least 150 population doublings after isolation from human tissue comprises providing a cell from the clone and allowing it to divide until it reaches senescence or 150 doublings.

13. the method of claim 1, wherein substantially all of said keratinocytes are free of a gross chromosomal abnormality.

14. The method of claim 1, wherein
the keratinocyte clone is isolated directly from the human tissue, and
the step of determining if the clone is capable of at least 150 population doublings after isolation from human tissue further comprises
(a) dividing the clone into at least two aliquots;
(b) performing serial passaging of the cells of one of the aliquots until the proliferative potential of said cells is exhausted or until said cells undergo 150 population doublings from the time of isolation from human tissue;
thereby determining if the clone is capable of at least 150 population doublings.

15. The method of claim 1, wherein the tissue is a skin sample; the keratinocyte clone is isolated from the skin sample prior to, or prior to a time sufficient for, two doublings from the time the sample is obtained; and determining if the clone is capable of at least 150 population doublings after isolation from human tissue comprises providing a cell from the clone and performing a cell division assay on said cell.

16. The method of claim 1, wherein the tissue is a skin sample; the keratinocyte clone is isolated from the skin sample directly from the skin sample without first passaging the cells; and determining if the clone is capable of at least 150 population doublings after isolation from human tissue comprises providing a cell from the clone and performing a cell division assay on said cell.

17. The method of claim 1, wherein the tissue is a skin sample; the keratinocyte clone is isolated directly from the skin sample without first passaging the cells; and determining if the clone is capable of at least 150 population doublings after isolation from human tissue comprises (a) dividing the clone into at least two aliquots; and (b) performing serial passaging of the cells of one of the aliquots until the proliferative potential of said cells is exhausted or until said cells undergo 150 population doublings from the time of isolation from human tissue.

18. A preparation of keratinocytes wherein substantially all of the colony-forming keratinocytes in the preparation are capable of at least 150 population doublings after isolation from human tissue.

19. The preparation of claim 18, wherein substantially all of the colony-forming keratinocytes in the preparation are free of a gross chromosomal abnormality.

20. A preparation of keratinocytes, wherein said preparation is made by a method comprising:
isolating at least one keratinocyte clone from a subject or from a human epithelial tissue;
determining if the clone is capable of at least 150 population doublings after isolation from human tissue;
optionally determining that the clone is free of a gross chromosomal abnormality;
thereby providing said preparation.
